(19) United States
(12) Patent Application Publication
(10) Pub. No.: US 2022/0296504 A1
(43) Pub. Date: Sep. 22, 2022

(54) INTRANASAL DELIVERY OF LEVODOPA POWDER BY PRECISION OLFACTORY DEVICE

(71) Applicant: Impel Pharmaceuticals, Inc., Seattle, WA (US)

(72) Inventors: John D. Hoekman, Seattle, WA (US); Kelsey H. Satterly, Seattle, WA (US); Inna Dashevsky, Seattle, WA (US); Aditya R. Das, Foster City, WA (US); Stephen B. Shrewsbury, Fallbrook, CA (US)

(21) Appl. No.: 17/498,381

(22) Filed: Oct. 11, 2021

Related U.S. Application Data
(63) Continuation of application No. 16/240,642, filed on Jan. 4, 2019, now abandoned.
(60) Provisional application No. 62/614,310, filed on Jan. 5, 2018.
(60) Provisional application No. 62/700,591, filed on Jul. 19, 2018.

(51) Int. Cl. A61K 9/00; A61P 25/16; A61M 15/00; A61K 31/198; A61K 9/16; A61K 31/197

(52) U.S. Cl. CPC A61K 9/0043; A61P 25/16; A61K 9/0053; A61M 15/0036; A61K 31/198; A61K 9/1652; A61K 31/197; A61M 15/003; A61K 9/1611; A61K 9/1623; A61M 15/009

(57) ABSTRACT

Methods are provided for treating OFF episodes in a patient with Parkinson's disease or a Parkinson syndrome, comprising administering to a subject with Parkinson's disease or a Parkinson syndrome experiencing an OFF episode an effective dose of a dry pharmaceutical composition comprising L-DOPA, wherein the dose is administered by an intranasal delivery device that provides, following intranasal administration, (a) a mean peak plasma levodopa concentration (Cₘₐₓ) of at least 200 ng/mL, with (b) a mean time to Cₘₐₓ (Tₘₐₓ) of levodopa of less than or equal to 60 minutes. Dry pharmaceutical compositions of levodopa suitable for intranasal administration and unit dosage forms comprising the dry pharmaceutical compositions are also provided.

### 1. CROSS-REFERENCE TO RELATED APPLICATIONS

This application is a continuation of U.S. application No. 16/240,642, which claims priority to U.S. provisional Application Nos. 62/700,591, filed Jul. 19, 2018, and 62/614,310, filed Jan. 5, 2018, each of which is incorporated herein by reference in its entirety.

### 2. BACKGROUND

In patients with Parkinson's disease, OFF episodes occur when levodopa (L-DOPA) levels are sub-therapeutic, and can occur at first waking in the morning or sporadically throughout the day. Rapid reduction in OFF episodes would provide improved quality of life and activities of daily living by allowing for more ON time.

However, existing treatments for OFF episodes are inadequate. While there are emerging alternatives for OFF episodes, these new alternatives may be suboptimal for various subsets of Parkinson's disease patients. For example, the FDA recently approved levodopa for oral inhalation (INBRIJA) for treatment of Parkinson disease OFF episodes. However, given common age-related comorbidities, dose-to-dose consistency may be difficult to achieve. Moreover, reported side effects include cough and upper respiratory tract infection in patients who have restricted mobility. Sublingual apomorphine, also in development, has the ability to resolve OFF episodes but may suffer from tolerability issues due to a high incidence of induced nausea, and may be difficult for patients to manage.

There is, therefore, a need for new methods of treating OFF periods in patients with Parkinson's disease.

### 3. SUMMARY

In a first aspect, methods are presented for treating OFF episodes in a patient with Parkinson's disease. The methods comprise administering to a subject with Parkinson's disease experiencing an OFF episode an effective dose of a dry pharmaceutical composition comprising L-DOPA, wherein the dose is administered by an intranasal delivery device that provides, following intranasal administration, (a) a mean peak plasma levodopa concentration (Cₘₐₓ) of at least 200 ng/mL, with (b) a mean time to Cₘₐₓ (Tₘₐₓ) of levodopa of less than or equal to 60 minutes. In particular embodiments, the mean peak plasma levodopa concentration (Cₘₐₓ) provided by the dose is at least 400 ng/mL.

In various embodiments, the dry pharmaceutical composition is a powder. In certain embodiments, the powder comprises L-DOPA in crystalline form. In certain embodiments, the powder comprises L-DOPA in non-crystalline, amorphous, form. In certain embodiments, the powder comprises L-DOPA in partially crystalline, partially amorphous form. In particular embodiments, the L-DOPA is an amorphous solid obtained by spray-drying.

In various embodiments, the dry pharmaceutical composition further comprises HPMC. In some embodiments, the dry pharmaceutical composition further comprises maltoside.

In typical embodiments, the method further comprises administering to the subject a peripherally-acting DOPA decarboxylase inhibitor (DDI). In specific embodiments, the (DDI) is administered orally.

Other features and advantages of the present disclosure will become apparent from the following detailed description, including the drawings. It should be understood, however, that the detailed description and the specific examples are provided for illustration only, because various changes and modifications within the spirit and scope of the invention will become apparent to those skilled in the art from the detailed description.

### 4. BRIEF DESCRIPTION OF THE DRAWINGS

FIG. 1 shows mean Plasma Concentration-Time Curves following intranasal administration of the indicated amounts of L-DOPA powder delivered by the nonhuman primate Precision Olfactory Delivery (“nhpPOD”) Device. The data were obtained in study number 2037-003, described in Example 1 below.

FIG. 2 shows mean Plasma Concentration-Time Curves following intranasal administration of 20 mg of L-DOPA (various formulations) delivered by nhpPOD Device in cynomolgus monkeys pre-dosed orally with the DOPA decarboxylase inhibitor, benserazide. The data were obtained in study 2037-004, described in Example 1 below. The 20 mg bulk L-DOPA (black line) data are drawn from Study 2037-003 and shown for comparison of the measured plasma levels in the absence of oral benserazide.

FIGS. 3A and 3B show mean Plasma Concentration-Time Curves following intranasal administration 20 mg L-DOPA (various formulations) delivered intranasally by the nhpPOD Device in monkeys pre-dosed with oral benserazide. The data were obtained in study 2037-006, as described in Example 1, with FIG. 3A plotting results without error bars, for clarity, and FIG. 3B including error bars. The line labeled “Bulk Sifted 20-40 μm L-Dopa” shows results from intranasal administration of bulk sifted L-DOPA with particles having diameters in the range of 20-40 μm (data from study 2037-004). The line labeled “Bulk L-Dopa API” shows results from intranasal administration of bulk L-DOPA (data from study 2037-003).

FIGS. 4A-4C show mean Plasma Concentration-Time Curves following intranasal administration of 20 mg L-DOPA (various formulations) delivered by the nhpPOD Device in monkeys pre-dosed with oral benserazide, from data obtained in study 2037-007 (Group 1-5 in Table 10), as described in Example 1, with FIG. 4A plotting results with error bars for all the PK time points (0-600 mins); FIG. 4B plotting results without error bars for clarity, for shorter PK time points (0-150 mins); and FIG. 4C plotting results without error bars, for even shorter PK time points (0-45 mins). FIGS. 4A-4C also provide data from previous studies for comparison, such as (i) 52F (Group 4 in Table 9), (ii) Bulk Sifted 20-40 um Crystalline L-Dopa (Group 2 in Table 7), (iii) 70A-L-Dopa:NaCl: HPMC:DSPC (68:2:16:14) (Group 1 in Table 9); and (iv) 70B-L-Dopa:NaCl: HPMC:DSPC (68:2:23:7) (Group 2 in Table 9).

FIGS. 5A-5E show Plasma Concentration-Time Curves for individual animals following 20 mg L-DOPA (various formulations) delivered by the nhpPOD Device in monkeys pre-dosed with oral benserazide, from data obtained in study 2037-007, as described in Example 1. FIG. 5A plots data for four individual animals in Group 1 (male 1001, male 1002, female 1501, female 1502); FIG. 5B plots data for four individual animals in Group 2 (male 2001, male 2002, female 2501, female 2502); FIG. 5C plots data for four individual animals in Group 3 (male 3001, male 3002, female 3501, female 3502); FIG. 5D plots data for four individual animals in Group 4 (male 4001, male 4002, female 4501, female 4502); and FIG. 5E plots data for four individual animals in Group 5 (male 5001, male 5002, female 5501, female 5502). Animals in each group were administered L-DOPA as provided in Table 9.

FIG. 6 illustrates an example nhpPOD device for administration of levodopa to non-human primates (NHPs).

FIG. 7A is an intranasal drug delivery device, in accordance with one or more embodiments.

FIG. 7B illustrates a partial cross-sectional view of the intranasal delivery device with removable tip attached, and a separate perspective view of the removable tip in its detached state, in accordance with one or more embodiments.

FIG. 7C is a perspective view of a tip and a capsule, in accordance with one or more embodiments.

FIG. 7D is a cross-sectional view of the tip and the capsule coupled to the device, in accordance with one or more embodiments.

FIG. 7E is an exploded view of the tip and the capsule, in accordance with one or more embodiments.

FIG. 7F is a perspective view of the tip with the capsule attached, in accordance with one or more embodiments

FIG. 7G is a cross-sectional view of the tip with the capsule attached, in accordance with one or more embodiments.

FIG. 7H is a cross-sectional view of the tip, in accordance with one or more embodiments.

FIG. 7I is a cross-sectional view of the tip, in accordance with one or more embodiments.

FIG. 7J is a cross-sectional view of an inlet interface of the tip with the capsule attached, in accordance with one or more embodiments.

FIGS. 7K-7N are perspective views of the tip of the device, in accordance with one or more embodiments.

FIG. 7O is a perspective view of the tip, in accordance with one or more embodiments.

FIG. 7P is a perspective view of the tip, in accordance with one or more embodiments.

FIG. 7Q is a perspective view of the tip coupled to the device, in accordance with one or more embodiments.

FIG. 7R is a cross-sectional view of the tip coupled to the device, in accordance with one or more embodiments.

FIG. 7S is a zoomed-in view of the inlet interface with the capsule attached, in accordance with one or more embodiments.

FIG. 7T is a perspective view of a second embodiment of a tip, in accordance with one or more embodiments.

FIG. 7U is a perspective view of the tip of FIG. 7T with a capsule attached, in accordance with one or more embodiments.

FIG. 7V is a perspective view of a puncture member, in accordance with one or more embodiments.

FIG. 7W is a perspective view of the puncture member, in accordance with one or more embodiments.

FIG. 7X illustrates a flow path of the second embodiment of the puncture member, in accordance with one or more embodiments.

FIG. 8 illustrates an example of a non-human primate precision olfactory delivery device, in accordance with one or more embodiments.

FIG. 9A illustrates another example of a non-human primate precision olfactory delivery device used in the studies 2037-003, 2037-004, 2037-006, 2037-007 described in Example 1, in accordance with one or more embodiments.

FIG. 9B illustrates a side view and a cross-sectional view of an actuator body of the intranasal device of FIG. 9A, in accordance with one or more embodiments.

FIG. 9C illustrates a side view of an extension tube of the intranasal device of FIG. 9A, in accordance with one or more embodiments.

FIG. 9D illustrates a zoomed-in view of two embodiments of a connecting interface at an end of the extension tube of FIG. 9C, in accordance with one or more embodiments.

FIG. 9E illustrates a side view and a cross-sectional view of a tip of the intranasal device of FIG. 9A, in accordance with one or more embodiments.

FIG. 10 graphs interim PK data from cohorts 1 and 2 of the human phase IIa clinical trial described in Example 2, in accordance with one or more embodiments.

### 5. DETAILED DESCRIPTION

5.1. Definitions

Unless defined otherwise, all technical and scientific terms used herein have the meaning commonly understood by a person skilled in the art to which this invention belongs.

An “OFF” episode is defined as a period during which a patient with Parkinson Disease (PD) or a Parkinson syndrome who is receiving an anti-Parkinson treatment has a UPDRS III motor score >30.

“Maltoside” refers to N-Dodecyl-β-D-maltopyranoside (n-dodecyl β-D-maltoside).

A pharmaceutical composition is “dry” if it has a residual moisture content of no more than 10%.

Intranasal administration of levodopa is “adjunctive to” an oral treatment with a decarboxylase inhibitor when levodopa is administered intranasally in sufficient temporal proximity to a prior oral administration of decarboxylase inhibitor that the plasma Cₘₐₓ of the intranasally administered levodopa is increased.

5.2. Other Interpretational Conventions

Particle sizes are sizes as reported by a Mastersizer 3000 laser diffraction particle size analyzer device (Malvern Panalytical).

Ranges: throughout this disclosure, various aspects of the invention are presented in a range format. Ranges include the recited endpoints. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Unless specifically stated or apparent from context, as used herein the term “or” is understood to be inclusive.

Unless specifically stated or apparent from context, as used herein, the terms “a”, “an”, and “the” are understood to be singular or plural. That is, the articles “a” and “an” are used herein to refer to one or to more than one (i.e., to at least one) of the grammatical object of the article. By way of example, “an element” means one element or more than one element.

In this disclosure, “comprises,” “comprising,” “containing,” “having,” “includes,” “including,” and linguistic variants thereof have the meaning ascribed to them in U.S. Patent law, permitting the presence of additional components beyond those explicitly recited.

Unless specifically stated or otherwise apparent from context, as used herein the term “about” is understood as within a range of normal tolerance in the art, for example within 2 standard deviations of the mean and is meant to encompass variations of ±20% or ±10%, more preferably ±5%, even more preferably ±1%, and still more preferably ±0.1% from the stated value.

5.3. Summary of Experimental Observations

We have conducted four single dose PK studies in the cynomolgus monkey to examine the PK following intranasal administration of multiple powder formulations of levodopa (L-DOPA) delivered using a handheld, manually actuated, metered-dose intranasal administration device adapted for use with non-human primates, the nhpPOD Device. The formulations examined included an unmodified crystalline powder (median particle size 50 μm), a sifted formulation containing crystalline L-DOPA particles with size range of 20-40 μm, and spray dried formulations with L-DOPA alone or containing NaCl with and without HPMC, 1,2-distearoyl-sn-glycero-3-phosphocholine (DSPC), or maltoside. We have found that spray-dried, amorphous, L-DOPA, formulated in a powder with HPMC and maltoside, when delivered intranasally to non-human primates with our intranasal delivery device, rapidly provides blood levels of levodopa above the level known to be correlated with improving OFF episodes in human patients.

Interim analysis of two of the cohorts enrolled in a phase IIa clinical trial in Parkinson disease patients demonstrated that a spray-dried formulation containing L-DOPA:NaCl:HPMC:Maltoside in the ratios 68:2:29:1 (wt %) delivered by a Precision Olfactory Delivery device was well tolerated. Interim pharmacokinetic data for cohort 1 (35 mg) and cohort 2 (70 mg) show that administration of a 70 mg dose reached blood concentrations in the range effective to treat OFF episodes with a mean time to Cₘₐₓ (Tₘₐₓ) of 30-60 minutes.

5.4. Methods of Treating Parkinson's Disease OFF Periods

Accordingly, in a first aspect, methods are provided for treating OFF episodes in a patient with Parkinson's disease or a Parkinson syndrome, comprising administering to a patient with Parkinson's disease or a Parkinson syndrome experiencing an OFF episode an effective dose of a dry pharmaceutical composition comprising levodopa (L-DOPA), wherein the dose is administered by an intranasal delivery device that provides, following intranasal administration, (a) a mean peak plasma levodopa concentration (Cₘₐₓ) of at least 200 ng/mL, with (b) a mean time to Cₘₐₓ (Tₘₐₓ) of levodopa of less than or equal to 60 minutes. In particular embodiments, the mean peak plasma levodopa concentration (Cₘₐₓ) provided by the dose is at least 400 ng/mL.

5.4.1. Patients

In the methods described herein, intranasal administration of levodopa is used to treat OFF episodes that occur despite oral administration of an anti-Parkinson treatment.

In typical embodiments, the intranasal administration of levodopa is adjunctive to oral administration of a DOPA decarboxylase inhibitor (“DDI”). In typical embodiments, the intranasal administration of levodopa is adjunctive to oral treatment with a DDI and oral treatment with levodopa. In some embodiments, the intranasal administration of levodopa is adjunctive to oral treatment with an oral dosage form containing a fixed dose combination of a DDI and levodopa. In various embodiments, the oral DDI is benserazide or carbidopa. In some embodiments, the oral DDI is benserazide. In some embodiments, the oral DDI is carbidopa.

In some embodiments, the patient has Parkinson's disease (“PD”).

In some embodiments, the patient has a Parkinson syndrome. In various embodiments, the Parkinson syndrome is selected from post-encephalitic parkinsonism, symptomatic parkinsonism following carbon monoxide intoxication, or symptomatic parkinsonism following manganese intoxication.

5.4.2. Effective Dose

In the methods described herein, the effective dose is a dose of levodopa effective to reverse an OFF episode within 60 minutes.

In some embodiments, the effective dose of levodopa is 25-150 mg or 35 -140 mg. In certain embodiments, the effective dose of levodopa is 35 mg, 70 mg, 105 mg, or 140 mg.

In some embodiments, the effective dose is administered as a single undivided dose. In some embodiments, the effective dose is administered as a plurality of equally divided sub-doses.

5.4.3. Dry Powder Composition

In various embodiments, the dry pharmaceutical composition is a powder.

In typical embodiments, the median diameter of the levodopa particle size distribution (D50) in the powder is 5 μm-500 μm. In some embodiments, the median diameter of the levodopa particle size distribution (D50) in the powder is 5 μm-250 μm, 5 μm-100 μm, 5 μm-75 μm, or 5 μm-50 μm. In certain embodiments, the median diameter of the levodopa particle size distribution (D50) in the composition is 10 μm-50 μm or 20 μm-40 μm.

Typically, the dry pharmaceutical composition comprises levodopa in a crystalline or amorphous form. In some embodiments, the dry pharmaceutical composition comprises levodopa in both crystalline and amorphous forms. In some embodiments, the dry pharmaceutical composition comprises levodopa in amorphous form. In particular embodiments, the amorphous levodopa is obtained by spray-drying.

In various embodiments, the dry pharmaceutical composition comprises no more than 80 wt % levodopa. In some embodiments, the composition comprises 50-80 wt % levodopa, 50-70 wt % levodopa, 65-70 wt % levodopa.

In various embodiments, the dry pharmaceutical composition further comprises a nonionic surfactant. In certain embodiments, the nonionic surfactant is an alkyl maltoside. In particular embodiments, the alkyl maltoside is n-dodecyl β-D-maltoside.

In some embodiments, the nonionic surfactant is present in the dry pharmaceutical composition at 0.1-10 wt %, more typically 1-5 wt %. In particular embodiments, the nonionic surfactant is present at 1 wt %.

In various embodiments, the dry pharmaceutical composition further comprises HPMC.

In various embodiments, the dry pharmaceutical composition further comprises a salt of a monovalent inorganic cation. Typically, the salt is NaCl. In some embodiments, the composition comprises 1-5 wt % NaCl, or 2-4 wt % NaCl.

In currently preferred embodiments, the dry pharmaceutical composition comprises 68 wt % levodopa, 2 wt % NaCl, 29 wt % HPMC, and 1 wt % n-dodecyl β-D-maltoside, and is a spray dried composition that comprises amorphous levodopa. In some embodiments, L-DOPA is spray dried in the presence of HPMC and/or maltoside. In other embodiments, HPMC and/or maltoside is added after spray drying of L-DOPA.

5.4.4. Device

In the methods described herein, the dose is administered by an intranasal delivery device that delivers a powder to the nasal cavity.

5.4.4.1. Nasal Drug Delivery Device

In various embodiments, the intranasal administration device is a nasal drug delivery device as described in U.S. Pat. No. 9,550,036, the disclosure of which is incorporated herein by reference in its entirety.

In some embodiments, the intranasal delivery device is a handheld, manually actuated, metered-dose intranasal administration device. In certain embodiments, the device is manually actuated, propellant-driven, metered-dose intranasal administration device. In particular embodiments, the dry pharmaceutical composition is, prior to device actuation, encapsulated within a capsule present within the device. In some embodiments, the dry pharmaceutical composition is stored within a dose container that is removably coupled to the device prior to device actuation. For example, the dose container may be inserted into a portion of the device or may be coupled to the device such that the dose container is in fluid communication with the device.

In various embodiments, the intranasal delivery device includes a housing body, a propellant canister housed within the housing body, a compound chamber containing a drug compound or designed to receive a drug compound, a channel in fluid communication with the propellant canister and the compound chamber, and an outlet orifice at a distal end of the channel. In this configuration, propellant released from the canister travels through the channel, contacts the drug compound in the compound chamber, and propels the drug compound out the outlet orifice for delivery into an upper nasal cavity.

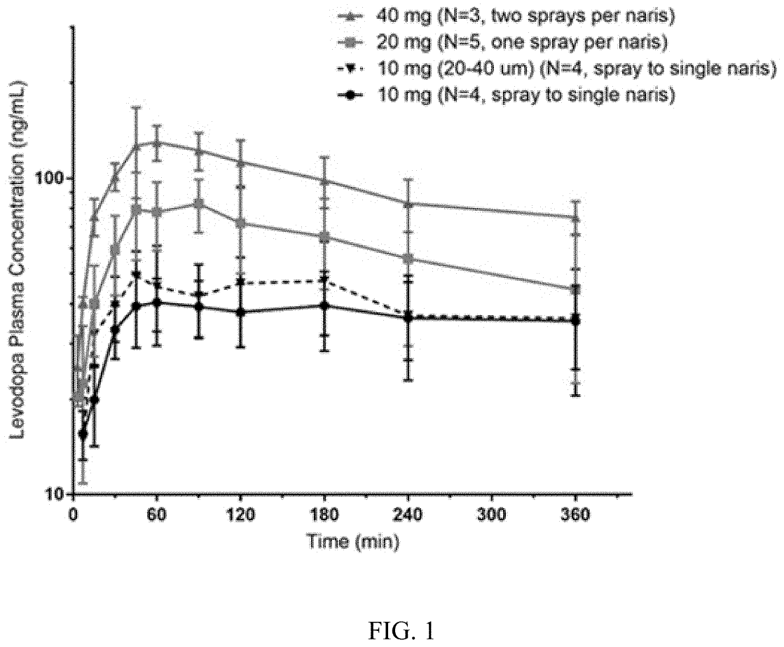

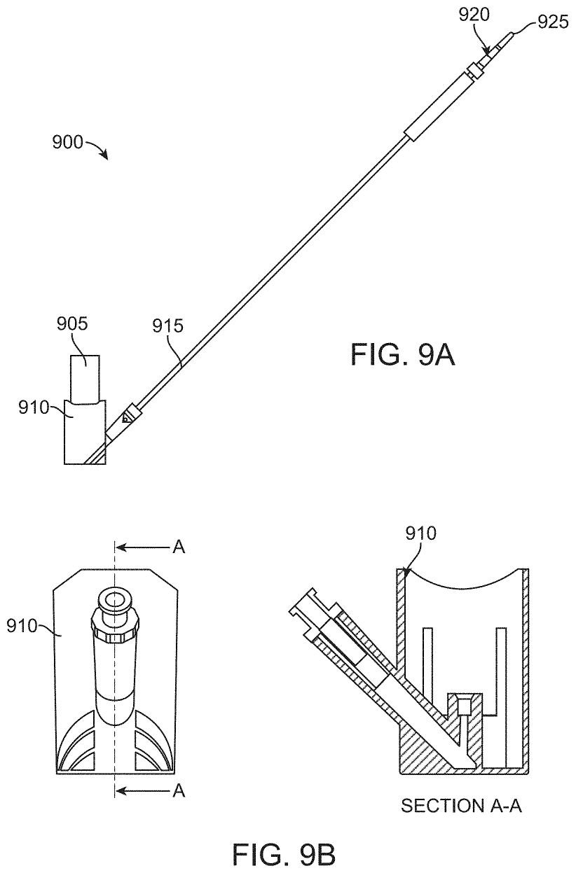

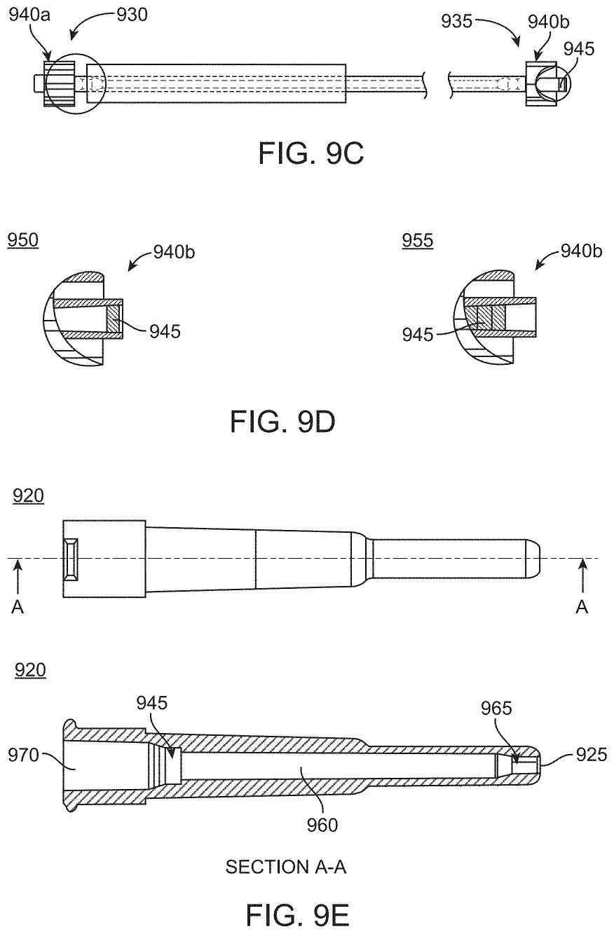

In various embodiments, the intranasal administration device is a non-human primate precision olfactory delivery (“nhpPOD”) device described in FIGS. 9A-E, also described in U.S. Pat. No. 9,550,036, incorporated by reference in its entirety herein. In one embodiment, the intranasal device is one of the embodiments of FIGS. 1, 2, and 9 of U.S. Pat. No. 9,550,036. In these embodiments, the drug compound is loaded directly into the compound chamber.

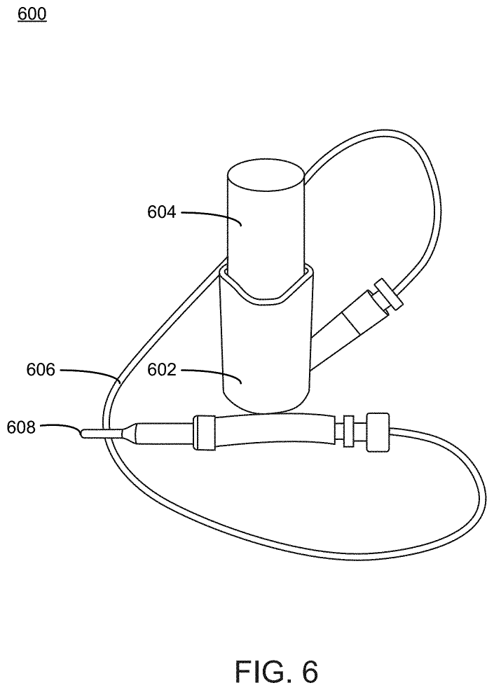

FIG. 6 illustrates an example nhpPOD device 600 for administration of levodopa to NHPs. Similar to the device embodiment described above, the device 600 includes a housing body 602, a propellant canister 604, a compound chamber (not shown), a channel 606, and an outlet orifice 608.

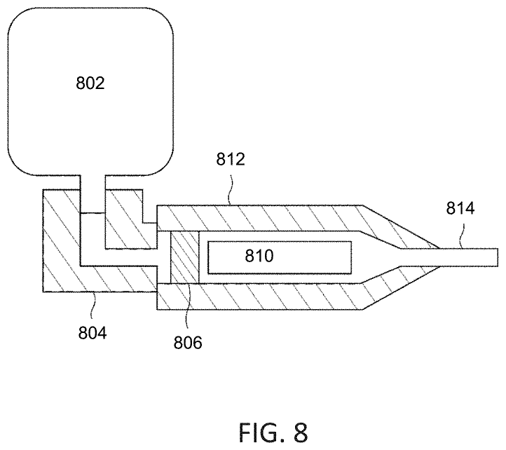

An additional embodiment of an nhpPOD device is shown in FIG. 8.

With reference to FIG. 8, a metered dose inhaler (MDI) canister 802 dispensing 25 μl hydrofluoroalkane is attached to the plastic actuator 804. The actuator is in gas communication with a polytetrafluoroethylene frit 806 which has a 50 μm pore size. The frit 806 is in communication with the dose holding cylinder 810 which is placed inside the body 812 of the POD in order to create an aerosolized flow. On actuation the HFA propellant 802 is converted to a gas by passing through the frit material 806 and then it mixes with the dose 810 and the dose and propellant mixture exits from the 23 gauge stainless steel tubing nozzle 814 which is covered with a fluorinated ethylene-propylene liner that was placed over the outside of the metal tip in order to protect the nasal epithelia from being damaged by the nozzle 814 during use. In one embodiment, the dose 810 is loaded directly into the body 812 without a holding cylinder.

5.4.4.2. Medical Unit Dose Container

In various embodiments, the intranasal administration device is a medical unit dose container as described in US 2016/0101245 A1, the disclosure of which is incorporated herein by reference in its entirety.

5.4.4.3. Intranasal Device with Inlet Interface

In various embodiments, the intranasal administration device is a medical unit dose container as described in US application Ser. No. 16/198,312, filed Nov. 21, 2018, the disclosure of which is incorporated herein by reference in its entirety, and repeated below for completeness.

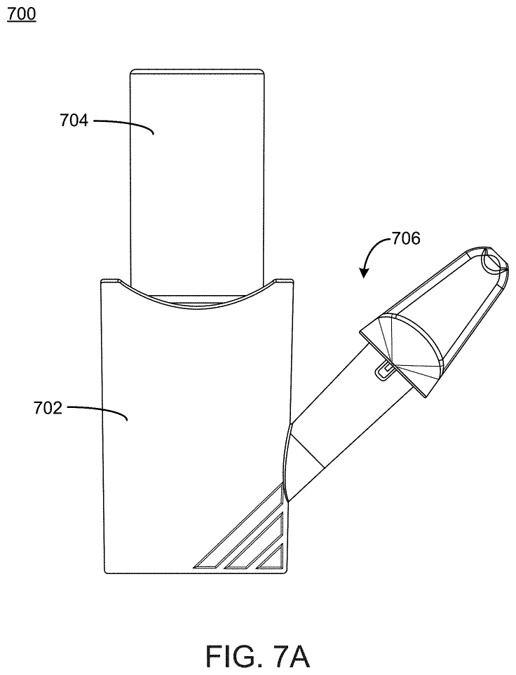

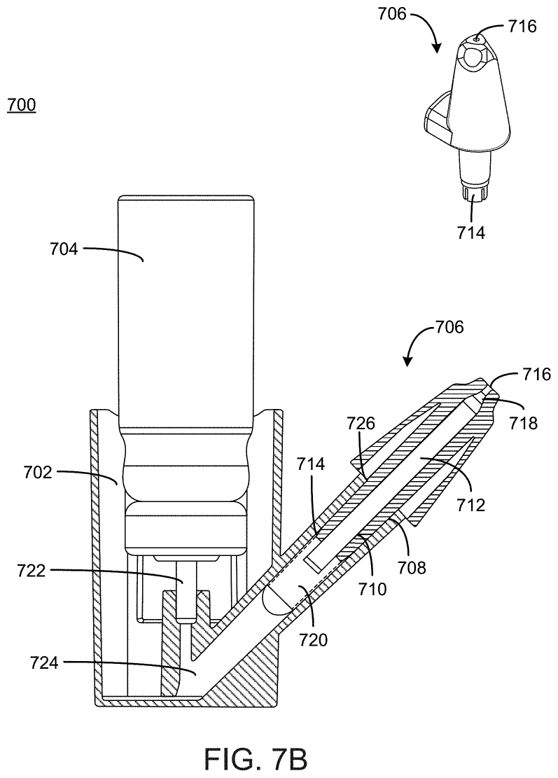

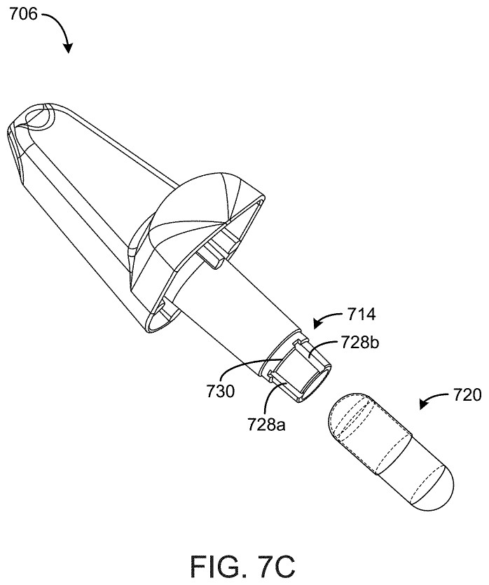

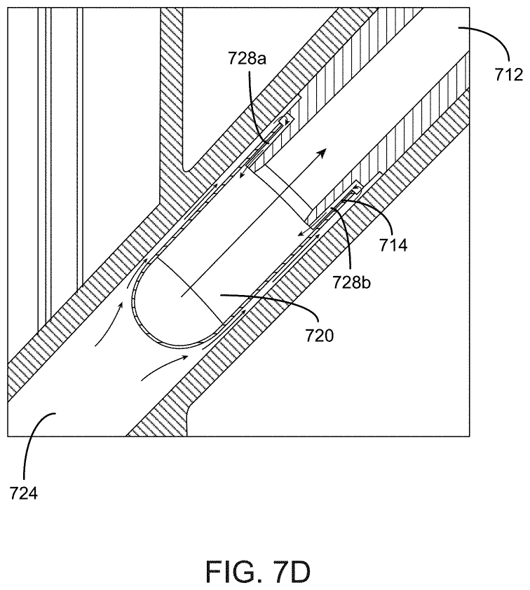

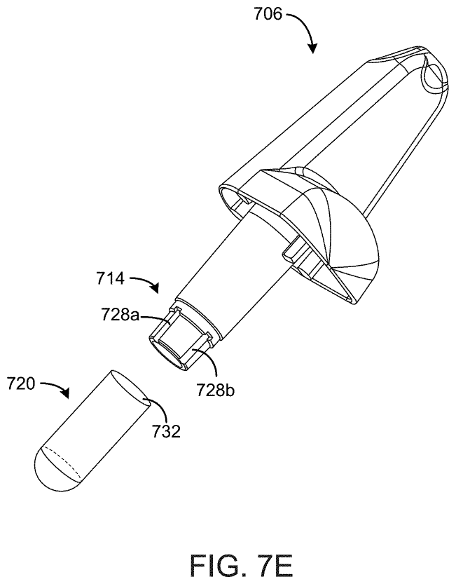

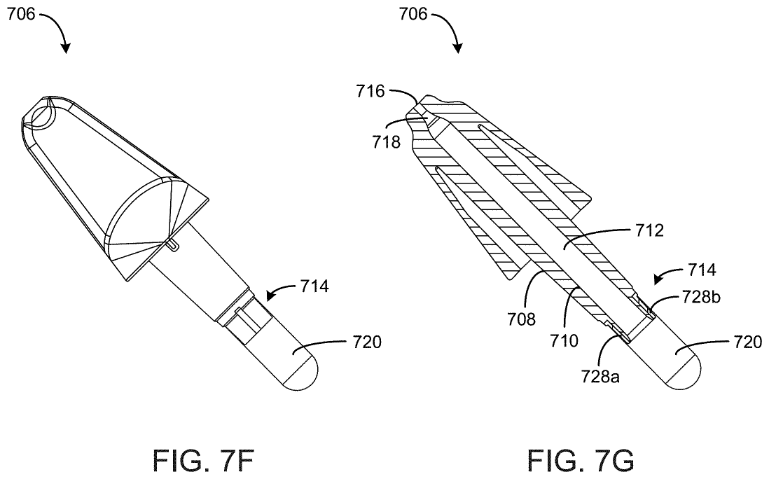

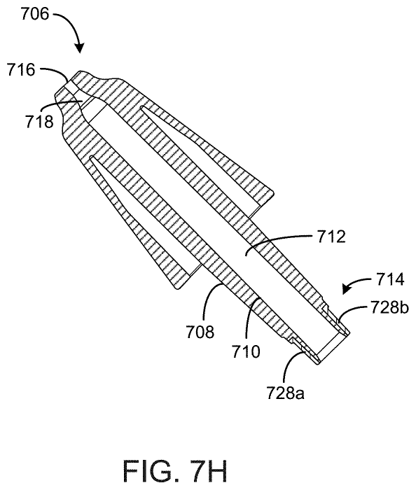

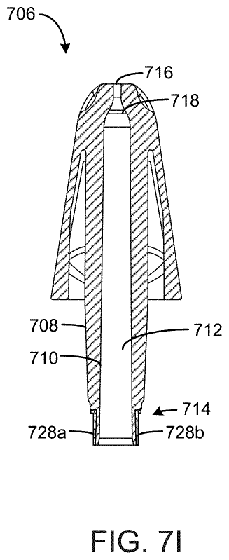

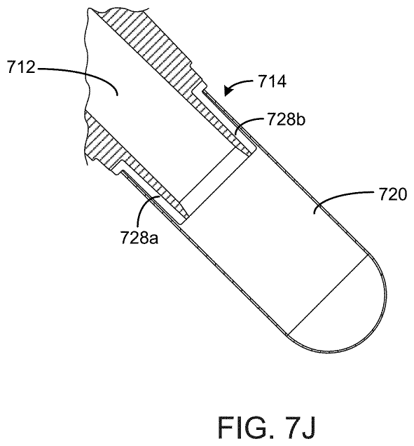

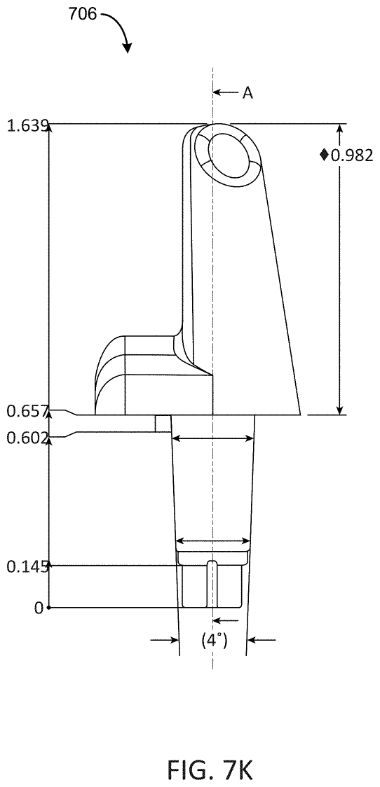

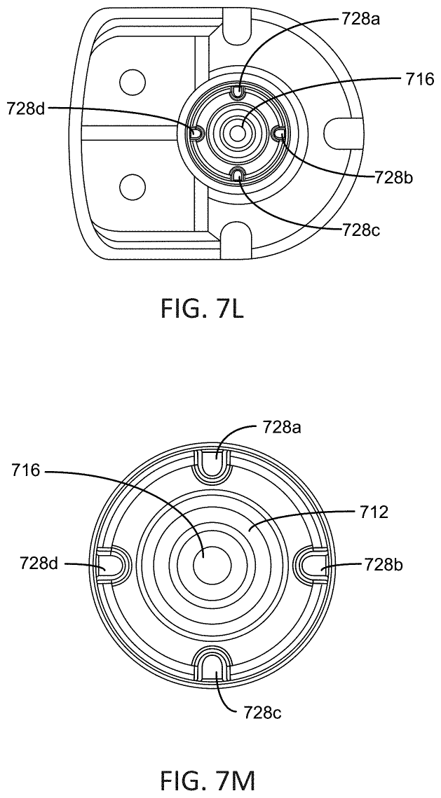

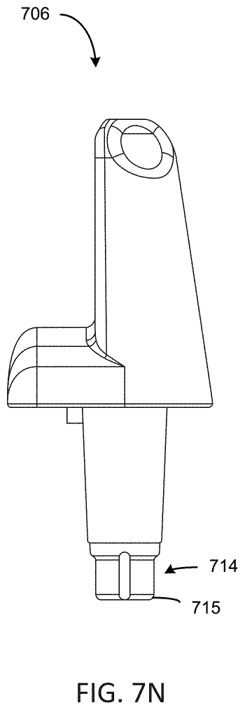

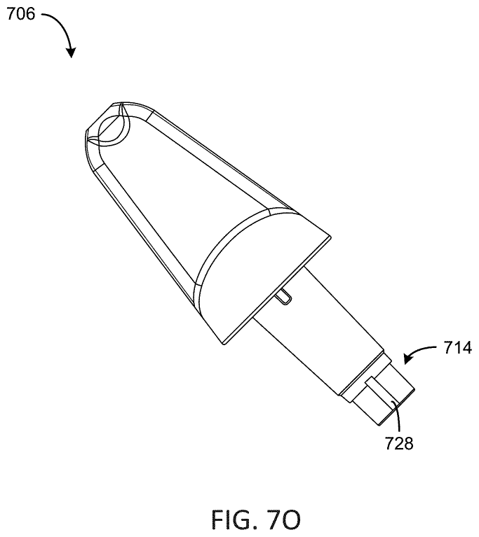

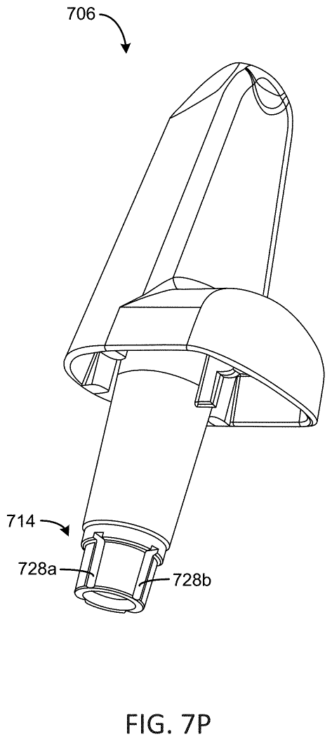

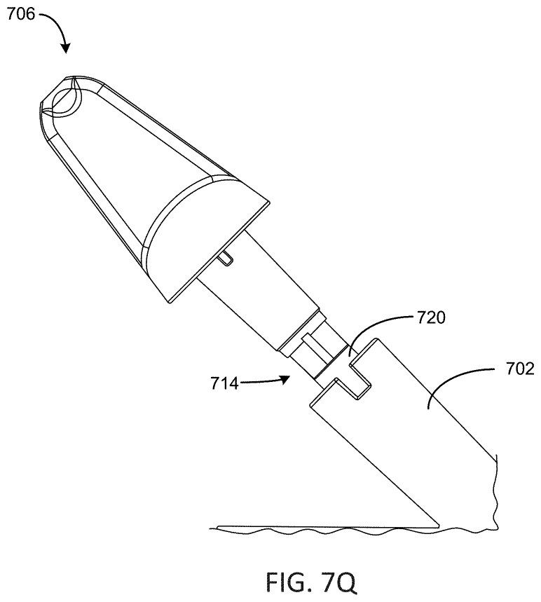

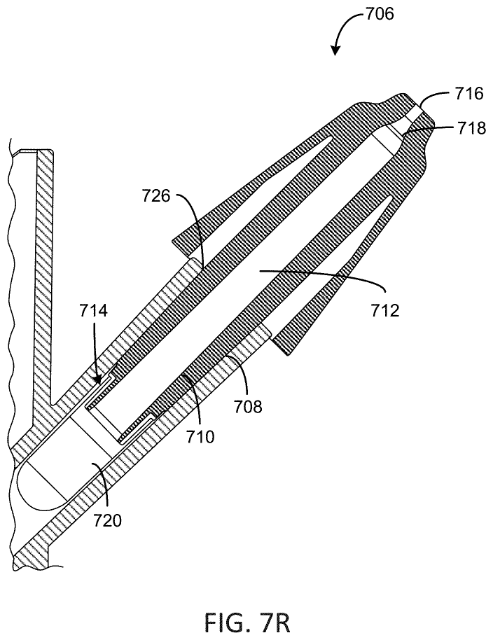

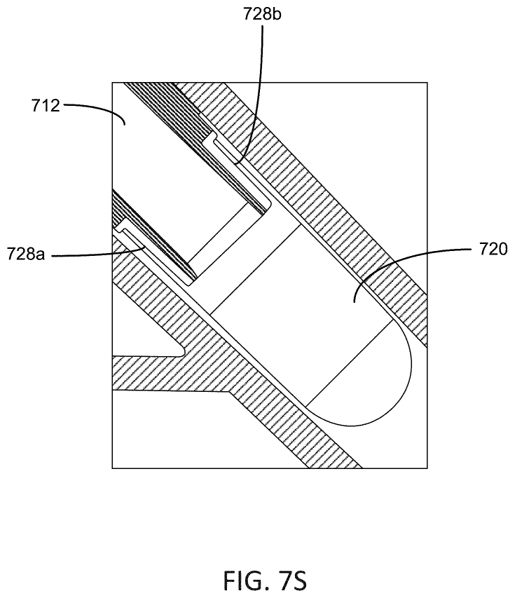

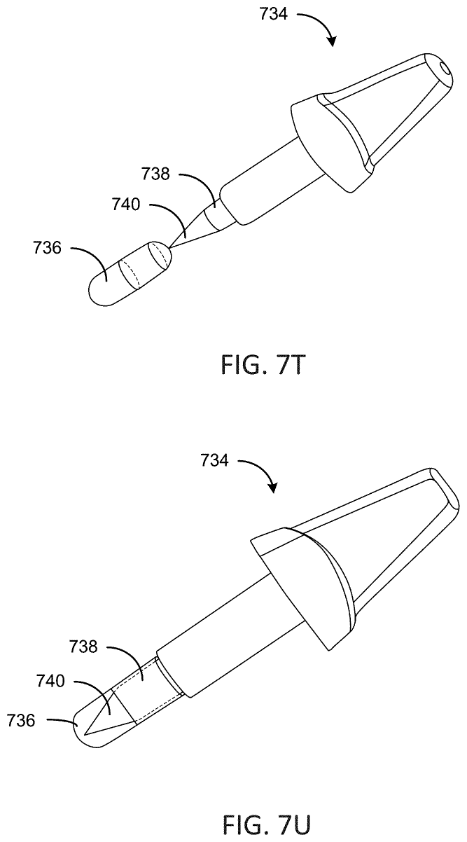

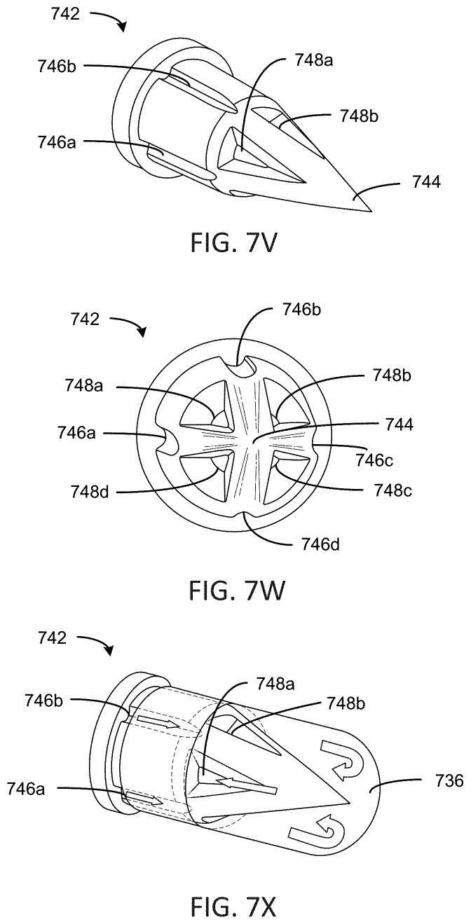

As shown in FIGS. 7A and 7B, the intranasal device 700 is designed to deliver a consistent mass of compound into the nasal cavity. For example, but not limited to, the compound may be an intranasal formulation in a powder form. The device 700 targets a specific region of the nasal cavity utilizing a narrow, targeted delivery plume. Specifically, the device 700 provides the compound to the upper one third of the nasal cavity. In one embodiment, the device 700 is used to administer the compound into the upper nasal cavity of a human. The upper nasal cavity includes the olfactory region and the middle and upper turbinate regions. In another embodiment, the device 700 is used to administer the compound into the upper nasal cavity of a non-human primate. The device 700 is also designed to simplify clinician loading of the compound into the device 700 and use thereof. The device 700 may be re-used to administer several doses of the compound.

FIG. 7B illustrates a partial cross-sectional view of the device 700 for delivering a compound intranasally. In the embodiment of FIG. 7B, the device 700 includes an actuator body 702, a propellant canister 704, and a tip 706. The tip 706 includes an outer wall 708 and an inner wall 710, an exit channel 712, an inlet interface 714, one or more grooves 728 (shown in FIG. 7C), an outlet orifice 716, and a nozzle 718. FIG. 7B illustrates the compound container 720 coupled to the inlet interface 714. The compound contained in the compound container 720 may be a liquid or a powder. In the embodiment of FIG. 7B, the compound is a powder.

As shown in FIG. 7B, the device 700 includes a propellant canister 704 positioned within the actuator body 702. The propellant canister 704 contains propellant. In one embodiment, the propellant may be pressurized. The propellant is a fluid, for example, a liquid or gas. In one aspect, the propellant is a liquid. In another aspect, the propellant is a gas. Propellants include pharmaceutically suitable propellants. Some examples of pharmaceutically suitable propellants include hydrofluoroalkane (HFA) including but not limited to HFA, HFA 227, HFA 134a, HFA-FP, HFA-BP and like HFAs. In one aspect, the propellant is liquid HFA. In another aspect, the propellant is gaseous HFA. Additional examples of suitable propellants include nitrogen or chloroflourocarbons (CFC). Additionally, propellants may be pressurized air (e.g. ambient air). The canister 704 may be a metered dose inhaler (MDI) device that includes a pressurized canister and metering valve 722 (including stem) to meter the propellant upon actuation. In one embodiment, a pump fitment (not shown) secures the metered valve 722 to the canister 704 and holds both components in place during device 700 use. One series of embodiments of the pump fitment consists of securing interfaces that retain the pump fitment within the actuator body 702, provide vertical displacement, and prevent rotation during installation of the canister 704.

The propellant canister 704 may have a capacity for distributing propellant for a certain number of doses. In one embodiment, the device 700 may be shipped without a canister 704 and the canister 704 may be loaded into the actuator body 702 by the user. In some embodiments, the propellant canister may be replaced with a new propellant canister, such that the device 700 may be reused. In one aspect, when the MDI device is actuated, a discrete amount of pressurized HFA fluid is released. The MDI may contain between about 30 to about 300 actuations, inclusive of endpoints, of HFA propellant. The amount of fluid propellant released upon actuation may be between about 20 microliters (μ1) and about 200 μl inclusive of endpoints, of liquid propellant.

The actuator body 702 comprises a propellant channel 724 that is in fluid communication with the propellant canister 704. The propellant channel 724 is in fluid communication with the inlet interface 714, which is configured to couple to the compound container 720 such that propellant released from the propellant canister 704 can be introduced into the compound container 720 via the one or more grooves 728 on the inlet interface 714. In the embodiment of FIG. 7B, the propellant channel 724 includes a port 726 at a distal end for receiving the tip 706. In this configuration, the tip 706 may be coupled and decoupled to the actuator body 702 by inserting the tip 706 into the port 726. In other embodiments, the port 726 may be inserted into the tip 706. In some embodiments, the port 726 and/or the tip 706 may include a sealing interface that creates an airtight seal between the propellant channel 724 and the tip 706 such that propellant released from the canister 704 does not escape out of the propellant channel 724 and is directed to the inlet interface 714.

The tip 706 may be coupled and decoupled to the actuator body 702, which enables a user to load and unload a compound container 720 to and from the inlet interface 714. The tip 706 includes the outer wall 708 and the inner wall 710, where the inner wall forms the exit channel 712 which extends between a proximal end and a distal end of the tip 706. The inlet interface 714 is positioned about a distal end of the outer wall 708, and the inlet interface 714 couples the compound container 720. In the embodiment of FIG. 7B, the inlet interface 714 is a collar that may be inserted into the compound container 720. In other embodiments, the inlet interface 714 may be a ring, band, port, or strap that interfaces with the compound container 720. The inlet interface 714 includes one or more grooves 728 (shown in FIG. 7C) for directing propellant released from the canister 704 into the compound container 720 coupled to the inlet interface 714. The released propellant then contacts the compound within the compound container 720, agitating and entraining the compound and propelling the compound through the exit channel 712 and out the outlet orifice 716 located at a distal end of the exit channel 712. In the embodiment of FIG. 7B, the tip 706 includes a nozzle at the distal end of the exit channel 712 for directing the released propellant and the compound out of the outlet orifice in a narrow plume.

FIG. 7C is a perspective view of the tip 706 and a compound container, in accordance with one or more embodiments. In the embodiment of FIG. 7C, the compound container 720 is a capsule. The capsule may be comprised of two portions fitted together. When separated, a portion of the capsule (e.g., a half-capsule, as shown in FIGS. 7E-7G) may be coupled to the tip 706. In use, the compound container 720 may contain a compound within the capsule. In one example, the compound is a powder. As shown in FIG. 7E, the half-capsule comprises an exit opening 732 of the compound container 720. The exit opening 732 may be coupled to the inlet interface 714, as shown in FIGS. 7F-7G. In the embodiments of FIGS. 7F-7G, the inlet interface 714 is inserted into the exit opening 732, and the compound container 720 may be secured to the inlet interface 714 via an interference fit. In an alternate embodiment, the exit opening 732 may be inserted into the inlet interface 714. As shown in FIGS. 7G-7H, the tip 706 has the outer wall 708 and the inner wall 710, where the exit channel 712 is formed by a bore or lumen through the inner wall 710. The exit opening 732 is fitted about the inlet interface 714 such that the compound container 720 and the exit channel 712 are in fluid communication.

As shown in FIGS. 7F, 7G, and 7J, the inlet interface 714 is, for example, a ring, band, port, collar, or strap interfacing with the compound container 720. As shown in FIGS. 7C, 7E, 7F, 7K, 7L, 7M, 7N, 7O, and 7P, one or more grooves 728 are positioned on the inlet interface 714 and create a flow path for the propellant released from the propellant canister 704 to travel into the compound container 720. An example of the grooves 728 includes but is not limited to channels, slots, radial ports, or passageways. The grooves 728 provide a pathway via the inlet interface 714 by which the propellant flows into the compound container 720. In one example, there are a plurality of grooves 728. The grooves 728 may be equally spaced about the inlet interface 714. The grooves 728 may be of equal size to each other or may be of differing sizes. The grooves 728 run along a length of the inlet interface 714 such that, when the compound container 720 is coupled to the inlet interface 714, a first portion of each groove 728 is exposed within the propellant channel 724 and a second portion of each groove 728 is positioned within the compound container 720. As shown in FIG. 7C, the inlet interface 714 includes a ledge 730 that is designed to abut the compound container 720 when coupled to the inlet interface 714 and the grooves 728 extend past the ledge 730 such that the grooves 728 are not fully covered by the compound container 720.

In use, as shown by the direction of the arrows in FIG. 7D, the propellant released from the canister 704 flows through the propellant channel 724 and into the compound container 720 via the grooves 728. The exit channel 712 is aligned with the exit opening 732 of the compound container 720. The propellant flows in the grooves 728 of the inlet interface 714, into the compound container 720 to agitate the powder, and the powder and the propellant exit the compound container 720 via the exit opening 732 congruent with the exit channel 712. The propellant and powder mixture are carried through the exit channel 712 through the nozzle 718 and exit the device 700 at the outlet orifice 716. In one example, the tip 706 may have one or a plurality of outlet orifices. The plume exiting the outlet orifice 716 has a narrow spray plume.

In one example of use of the device 700, at time of use, a user separates a pre-filled capsule into its two halves. In one example, the capsule is prefilled with a powder compound. The half-capsule is coupled to the tip 706 via the inlet interface 714. As shown in FIGS. 7P and 7Q, the tip 706 is then coupled to the actuator body 702. A propelling gas, for example from either a refrigerant or compressed gas source, is directed through the propellant channel 724 and towards the filled powder capsule. The grooves 728 around the inlet interface 714 of the tip 706 introduce high velocity jets of propellant gas which agitate the dry powder into a suspension within the propellant gas (data not shown but confirmed with high speed close up video). Grooves 728 that introduce gas tangentially to the semispherical-shaped bottom of the compound container 720 creates jets which enhance stirring and entrainment of powder. Once the powder has been suspended, it is evacuated through the exit opening 732, into the exit channel 712, and out the outlet orifice 716 of the device 700.

Generally, when accelerating a powder formulation through a restricting orifice, any constricting junction will cause the powder to clog. Since the powder administered by this device 700 is suspended within the propellant gas prior to evacuation, it can be further throttled and directed without device clogging. As a result, a much larger mass of powder can be delivered through a much smaller outlet orifice without the device 700 being prohibitively long. The time from propellant actuation to end of compound delivery is less than 1 second.

The grooves 728 in the proximal end of the tip 706 promote gas flow into the compound container 720. In one example, the HFA gas is directed (e.g. orthogonally or near-orthogonally) at the surface of the powder dose residing in the compound container 720, which creates rapid agitation and entrainment of the powder. The semispherical shape of the compound container 720 promotes gas redirection to the exit channel 712 of the tip 706 as shown in FIG. 7D. The arrows of FIGS. 7B and 7D show the direction of propellant flow after the device 700 has been actuated.

The actuator body 702 attached and seals to the propellant canister 704 and the tip 706, creating a pressurized flow path for the propellant gas. In certain aspects, the actuator body 702 is a reusable component. In certain aspects, the canister 704 is a reusable component.

In one example, the compound container 720 is a standard Size 3 drug capsule, although one of skill in the art would know how to use other sized drug capsules and modify the device 700 to fit same. Additionally, in another example, the compound container 720 may not be a capsule, but another container capable of containing a compound, such as but not limited to an ampoule. In one example, the ampoule may be made of plastic, and in one example it may be a blow fill sealed ampoule. To load the device 700, the user or clinician will separate a prefilled formulation containing capsule, discard the cap, and install the capsule over the tip 706. An empty compound container 720 can also be filled by a clinician at time of use before installing the compound container 720 onto the tip 706. In certain examples, the capsule is a disposable component.

The tip 706 receives the compound container 720 during loading and is then coupled to the actuator body 702 prior to use. When the propellant canister 704 is actuated, expanding propellant gas is introduced into the compound container 720 via the grooves 728 around the inlet interface 714 of the tip 706. The resulting propellant gas jets agitate and entrain the powder formulation within the compound container 720, which then exits through the exit channel 712 and the outlet orifice 716 of the tip 706. In one example, the tip 706 is a disposable component. FIG. 7K illustrates example measurements of the tip 706 with units in inches. In the embodiment of FIG. 7N, the inlet interface 714 may include a radius along a bottom edge 222 to aid placement of the compound container 720 onto the tip 706. The radius of curvature may range between approximately 0.007 inches to 0.027 inches, inclusive.

FIGS. 7T and 7U illustrate perspective views of a second embodiment of a tip 734. Similar to the tip 706, the tip 734 may be coupled and decoupled to the actuator body 702, which enables a user to load and unload a compound container 736 to and from the tip 734 for delivery to an upper nasal cavity of a user using the device 700. As shown in FIGS. 7T and 7U, a compound container 736 is a capsule. The compound container 736 may, in one example, contain a powder. In the embodiments of FIGS. 7T and 7U, the tip 734 includes an inlet interface 738 for coupling the compound container 736, where the inlet interface 738 has a puncture member 740. The puncture member 740 is designed to puncture the compound container 736 to create an opening in the compound container 736. The puncture member 740 may comprise a sharp point, a sharp angle, a blade-like edge, or other suitable geometries for puncturing the compound container 736. In one embodiment, the inlet interface 738 includes more than one puncture member 740, where each puncture member 740 is designed to puncture the compound container 736. The puncture members 740 may be positioned about the inlet interface 738 in a pattern, symmetrically, or at random. In one example, in use, a user may remove the tip 734 from the actuator body 702, load the compound container 736 into the port 726 of the propellant channel 724, and then insert the tip 734 back into the port 726. As the tip 734 is coupled to the actuator body 702, the puncture member 740 punctures the capsule. In this configuration, the punctured capsule fits around the puncture member 740, as shown in FIG. 7U. In alternate embodiments, the puncture member 742 may comprise a plurality of puncture points 744 that each puncture the compound container 736. The plurality of puncture points 744 may be spaced about the puncture member 742.

FIGS. 7V and 7W illustrate perspective views of a puncture member 742 that may be used with the tip 734, in accordance with one or more embodiments. As shown in FIG. 7V, the puncture member 742 may be a collar, ring, band, port or strap that couples with the punctured compound container 736. The puncture member 742 includes one or more puncture grooves 746 that, similar to grooves 728, form a flow path between the propellant channel 724 and the compound container 736. The propellant from the propellant canister 704 enters via the one or more puncture grooves 746 of puncture member 742 and flows along the puncture grooves 746 and into the punctured compound container 736. As shown in FIGS. 7V and 7W, the puncture member 742 includes a plurality of puncture openings 748. In the embodiments of FIGS. 7V, 7W, 7X, the puncture openings 748 are in fluid communication with the exit channel 712. The propellant from the propellant canister 704 flows into the puncture grooves 746, mixes with the powder in the punctured compound container 736, and flows into the puncture openings 744 to the exit channel 712. The arrows of FIG. 7X illustrate the flow path of the propellant. The exit channel 712 provides a route for the propellant and the powder to the nozzle 718 and the outlet orifice 716. The mixture of propellant and powder exit the device 700 via the outlet orifice 716. The plume exiting the device 700 is a narrow spray plume. In this embodiment, the puncture member 742 may be integrally molded as a single piece or may consist of two or more pieces. In one example, the puncture member 742 may be a separately molded piece acting in association with the inlet interface 738 (where the capsule attaches). In some embodiments, an inlet interface may include more than one puncture member 742.

As shown in FIGS. 7V and 7W, as an alternate to the capsule being manually separated prior to placement on the tip 734, the tip 734 may include an integrated puncture member 742 and puncture grooves 746. In order to create a repeatable puncture of the compound container 736, a puncture member 742 comes to a single point, puncture point 744. In one example, the puncture point 744 includes puncture openings 746 that are radially spaced about the puncture point 744. The puncture openings 746 are in fluid communication with the exit channel 712 for the powder to be evacuated from the compound container 736.

As shown in FIG. 7X, by allowing the propellant flow path to be created with an inline puncture motion, loading the compound container 736 onto the tip 734 is simplified for the user, as the compound container 736 does not require manual manipulation and separation. In one example, the puncture member 742 is formed integrally with the tip 734. In one example, the filled compound container 736 may be filled and installed into either the actuator body 702 or the tip 734 during manufacturing of the device 700. At time of use, a user may apply a linear motion to drive the puncture member 742 into the pre-filled compound container 736, creating a complete gas flow path for dosing prior to propellant actuation.

The invention is further described in the following examples, which are not intended to limit the scope of the invention.

Powder Capsule

In one embodiment, a device was constructed and tested. Testing was conducted for residual powder in the compound container after actuation. The device has equivalent performance of powder delivery, as determined by residuals after actuation, when 2 or more but less than 6 grooves on the inlet interface are used. In this example, the grooves are in combination with 63 mg of HFA propellant and a 0.040″ outlet orifice of the nozzle. Four grooves (every 90 degrees) were found to provide uniform gas delivery.

Dose Mass

Dose mass reproducibility testing was conducted. The standard deviation on dose delivery shows the device is capable of delivering consistent dose masses. The mean residual of dose left in the device was <5%, showing very little dose is lost in the device.

TABLE A
Mass reproducibility of final molded device

| | |
|---|---|
| n | 49 |
| Mean (mg) | 34.9 |
| Standard Deviation (mg) | 1.0 |
| Min (mg) | 32 |
| Max (mg) | 36.7 |
| Range | 4.7 |
| Mean % Residual | 3.8% |

5.4.4.4.Intranasal Device with Plurality of Frits

FIG. 9A illustrates another example of a non-human primate precision olfactory delivery device 800 used in the study 2037-003, 2037-004, 2037-006, 2037-007, and FIG. 9B illustrates a side view and a cross-sectional view of an actuator body 910 of the intranasal device 900 of FIG. 9A. The device 900 may deliver a compound that is a liquid, a powder, or some combination thereof. The device 900 includes a propellant canister 905, the actuator body 910, an extension tube 915, and a tip 920. Similar to the device 1, the propellant canister 905 is in fluid communication with the actuator body 910 such that propellant released from the propellant canister 905 travels through the actuator body 910, through the extension tube 915, through the tip 920, and out an exit opening 925 of the tip 920. A compound may be loaded into the tip 920 such that as the propellant travels through the tip 920, the propellant contacts the compound and propels the compound to the exit opening 925, where the propellant and compound exit as a plume.

FIG. 9C illustrates a side view of the extension tube 915 of the intranasal device 900 of FIG. 9A. The extension tube 915 is a tube comprising an internal channel that creates fluid communication between the actuator body 910 and the tip 920. In the embodiments of FIGS. 9A to 9D, a first end 930 of the extension tube 915 couples to the actuator body 910 and a second end 935 of the extension tube 915 couples to the tip 920 each via a respective connecting interface 940a, 940b (collectively referred to as “940”). The connecting interface 940 comprises a luer lock having a male or a female end on each side of the luer lock. In the embodiment of FIGS. 9A to 9D, each connecting interface 940 comprises a luer lock having two male ends. Accordingly, the male ends of the connecting interface 940a insert into the actuator body 910 and the first end 930, respectively, and the male ends of the connecting interface 940b insert into the tip 920 and the second end 935, respectively. As illustrated in FIG. 9C, the second end 935 may include a plurality of frits 945 positioned within an internal channel of the luer lock. A frit 945 may be configured to convert a liquid propellant into a gas as the propellant passes through the frit 945. Alternatively, the extension tube 915 in FIG. 9B can be configured to convert liquid propellant into a gas. The frit 945 may be composed of porous material. The number of frits 945 may vary in different embodiments. As the number of frits increases, the strength of the plume may be reduced, for example, in terms of its impact force, velocity, plume width, other similar metrics, or some combination thereof. Similarly, the length of the extension tube 915 may be adjusted such that the propellant has a longer or shorter distance to travel through. Calibrating the strength of the plume may enable the device 900 to accurately deliver the compound to the nasal cavity. FIG. 9D illustrates a zoomed-in view of the connecting interface 940b at the second end 935 of the extension tube 915 of FIG. 9C—a first example embodiment 950 includes a single frit 945, and a second example embodiment 955 includes three frits 945 stacked in succession. The number of frits 945 may be selected based on the type of compound. For example, a single frit 945 may be used for a powder compound, while three frits 945 may be used for a liquid compound, or vice versa.

FIG. 9E illustrates a side view and a cross-sectional view of the tip 920 of the intranasal device of FIG. 9A. The tip 920 is designed to be inserted into a nasal opening. The tip 920 comprises an internal channel 960 and the exit opening 925 for delivering the compound to the nasal cavity. In the embodiment of FIG. 9E, the tip 920 comprises a frit 945 seated within the internal channel 960. The frit 945 may be configured to convert a liquid propellant into a gas as the propellant passes through the frit 945. The frit 945 may be composed of porous material. In the embodiment of FIG. 9E, tip 920 further comprises a nozzle 965 at a distal end of the tip 920 near the exit opening 925. The nozzle 965 may enhance deposition of the compound within the nasal cavity, such as to the upper olfactory region of a user. In some embodiments, the nozzle 965 may include a single orifice, and, in alternate embodiments, the nozzle 965 may include a plurality of orifices (e.g., between 2 to 11 orifices). In some embodiments, the tip 920 may not include a nozzle. Different embodiments of tips may be used based on different types of compounds to be delivered to the nasal cavity of the user. For example, a tip for delivering a powder compound may not include a nozzle, while a tip for delivering a liquid compound may include a nozzle, or vice versa. In addition, the number of orifices in the nozzle may similarly vary based on the type of compound. A compound may be loaded into the tip 920 such that the compound is contained within the internal channel 960. In the embodiment of FIG. 9E, the compound is loaded into the tip 920 through an opening 990 at a proximal end of the tip 920 before the frit 945 is seated within the internal channel 960. The frit 945 is then inserted to contain the compound inside the tip 920. In an alternate embodiment, for example an embodiment in which the tip 920 does not include a nozzle 965, the compound may be loaded into the tip through the exit opening 925. In the configuration of FIG. 9E, the propellant travels from the propellant canister 905, through the actuator body 910 and extension tube 915, through the tip 920 and contacts the frit 945, and then contacts the compound within the internal channel 960, propelling the compound through the exit opening 925, where the propellant and compound exit as a plume that is delivered within the nasal cavity of the user.

5.5. Dry Pharmaceutical Composition

In another aspect, dry pharmaceutical compositions suitable for intranasal administration are provided. The compositions comprise levodopa, and at least one excipient.

In typical embodiments, the dry pharmaceutical composition is a powder.

In some embodiments, the median diameter of the levodopa particle size distribution (D50) in the powder is 5 μm-500 μm, 5 μm-250 μm, 5 μm-100 μm, or 5 μm-75 μm. In some embodiments, the median diameter of the levodopa particle size distribution (D50) in the powder is 5 μm 50 μm, 10 μm-50 μm, or 20 μm-40 μm.

In various embodiments, the composition comprises levodopa in a crystalline or amorphous form. In some embodiments, the composition comprises levodopa in amorphous form. In some embodiments, the composition comprises levodopa in a partially crystalline and partially amorphous form. In certain embodiments, the amorphous levodopa is obtained by spray-drying. In some embodiments, the composition comprises levodopa in a crystalline form and an amorphous form.

In various embodiments, the dry pharmaceutical composition comprises no more than 85 wt % levodopa, or no more than 80 wt % levodopa. In certain embodiments, the composition comprises 50-80 wt % levodopa, 50-70 wt % levodopa, or 65-70 wt % levodopa.

In typical embodiments, the dry pharmaceutical composition further comprises a nonionic surfactant. In some embodiments, the nonionic surfactant is an alkyl maltoside, and in currently preferred embodiments, the alkyl maltoside is n-dodecyl β-D-maltoside.

In some embodiments, the nonionic surfactant is present at 0.1-10 wt %, more preferably, 1-5 wt %. In particular embodiments, the nonionic surfactant is present at 1 wt %.

In various embodiments, the dry pharmaceutical composition further comprises hydroxypropyl methyl cellulose (HPMC).

In various embodiments, the dry pharmaceutical composition further comprises a salt of a monovalent inorganic cation. In typical embodiments, the salt is NaCl. In certain embodiments, the composition comprises 1-5 wt % NaCl or, more preferably, 2-4 wt % NaCl.

In currently preferred embodiments, the dry pharmaceutical composition comprises 68 wt % levodopa, 2 wt % NaCl, 29 wt % HPMC, and 1 wt % n-dodecyl β-D-maltoside. In particularly preferred embodiments, the composition is a spray dried composition that comprises levodopa in amorphous form.

5.6. Unit Dosage Form

In another aspect, unit dosage forms are provided. The unit dosage form contains a dry pharmaceutical composition as described in Section 5.4 above.

In typical embodiments, the unit dosage form contains 25-150 mg of levodopa. In certain embodiments, the unit dosage form contains 35 -140 mg of levodopa. In particular embodiments, contains 35 mg of levodopa or 70 mg of levodopa.

In some embodiments, the unit dosage form is a capsule that encapsulates the dry pharmaceutical composition. In certain embodiments, the capsule is a hard capsule. In particular embodiments, the hard capsule is an HPMC hard capsule.

In some embodiments, the unit dosage form is a dose container that is configured to be removably coupled to an intranasal delivery device. In particular embodiments, the dose container is a tip that is configured to be removably coupled to an intranasal delivery device.

5.7. Experimental Examples

The invention is further described through reference to the following experimental examples. These examples are provided for purposes of illustration only, and are not intended to be limiting.

5.7.1. Example 1: Non-Human Primate PK Studies

A series of powder formulations of L-DOPA (levodopa) were developed and manufactured to assess the pharmacokinetics of intranasal administration of levodopa in non-human primates (“NHP”). The goal of the powder formulation development was to obtain a formulation that, following intranasal delivery using a non-human primate Precision Olfactory Delivery (“nhpPOD”) Device, would result in a rapid plasma concentration increase to >200 ng/mL, preferably >400 ng/mL, such that the formulation would be expected to positively impact “OFF” episodes in Parkinson's disease.

Four single dose PK studies in the cynomolgus monkey were performed to examine the PK following administration of multiple powder L-DOPA formulations delivered by the intranasal route using the nhpPOD Device. The formulations examined included an unmodified crystalline powder (median particle size of about 50 μm), a sifted formulation containing crystalline L-DOPA particles with a defined size range of 20-40 μm, and spray dried formulations with L-DOPA alone or containing NaCl with and without HPMC, 1,2-distearoyl-sn-glycero-3-phosphocholine (DSPC), or maltoside. The placebo control, also delivered intranasally by the nhpPOD Device, was mannitol or microcrystalline cellulose (“MCC”). The formulations were delivered in the presence or in the absence of oral benserazide, a dopamine decarboxylase inhibitor.

Specifically, in the first single dose PK study (“2037-003”), a micronized crystalline levodopa powder (median particle size of about 50 μm) was administered without oral pretreatment of the animal with benserazide. In the second single dose PK study (“2037-004”), spray dried formulations of L-DOPA were administered in the presence of oral benserazide. In the third single dose PK study (“2037-006”), spray dried L-DOPA formulations including L-DOPA, NaCl, HPMC, maltoside, and/or DSPC, were administered in the presence of oral benserazide. In the fourth single dose PK study (“2037-007”), spray dried levodopa formulations from a second contract research organization that included maltoside at different concentrations (0.1, 0.5, 1%) were administered in the presence of oral benserazide. In each study, Cₘₐₓ and Tₘₐₓ were measured and compared to the value measured in other studies. Table 1 summarizes specific experimental conditions for each study.

TABLE 1
PK study designs

| | Study | Study | Study | Study |
|---|---|---|---|---|
| | 2037-003 | 2037-004 | 2037-006 | 2037-007 |
| Drug | Micronized | Spray dried | Optimized spray | Optimized spray |
| | crystalline | levodopa, | dried levodopa | dried levodopa |
| | levodopa | amorphous and | (CRO = Bend | (CRO = Hovione), |
| | (D₅₀ = 54 μm) | crystalline | Research), | amorphous and |
| | | polymorphs | amorphous and | crystalline |
| | | | crystalline | characteristics, |
| | | | polymorphs with | 0.1%, 0.5% and 1% |
| | | | combinations of | maltoside tested |
| | | | HPMC, DSPC and | along with |
| | | | maltoside | formulations from |
| | | | | different |
| | | | | manufacturing |
| | | | | processes |
| Dose | 10, 20, 40 mg | 20 mg (all groups) | 20 mg (all groups) | 20 mg (all groups) |
| Device | nhpPOD Device | nhpPOD Device | nhpPOD Device | nhpPOD Device |
| | #1, higher impact | #2, lower impact | #2, lower impact | #2, lower impact |
| | force | force | force | force |
| Administration | Single dose, | Single dose, | Single dose, | Single dose, |
| | Awake | Awake | Awake | Awake |
| DDI^{a} | None used | All groups: 5 mg | Groups 1 to 4: 5 mg | All groups: 5 mg |
| | | oral benserazide | oral benserazide | oral benserazide |
| | | at −24, −16, −8, | at −24, −16, −8, | at −24, −16, −8, |
| | | −0.75 hr | −0.75 hr | −0.75 hr |
| Plasma | 3, 7, 15, 30, 45, 60, | 3, 7, 15, 30, 45, 60, | 3, 7, 15, 30, 45, 60, | 3, 7, 15, 30, 45, 60, |
| PK Time | 90, 120, 180, 240, | 90, 120, 240, 360, | 90, 120, 240, 360, | 90, 120, 240, 360, |
| Points | 360 | 600 | 600 | 600 |
| Analysis | LC/MS/MS | Same LC/MS/MS | Same LC/MS/MS | Same LC/MS/MS |
| | | assay, also | assay as study #2 | assay as study #2 |
| | | assessed | (2037-004) | (2037-004) |
| | | benserazide | | |
| | | interference | | |

^{a}DDI refers to a peripherally-acting dopa decarboxylase inhibitor.
^{b}n-Dodecyl β-D-maltoside (“DDN”) was used as maltoside.

notes:

5.7.1.1. Single Dose Intranasal Pharmacokinetic Study in the Cynomolgus Monkey (non-GLP, Research Study Number 2037-003)

A single dose PK study was performed in the cynomolgus monkey. Crystalline levodopa (L-DOPA) dry powder, manufactured by Teva, was administered intranasally using an nhpPOD Device (non-human primate Precision Olfactory Delivery Device). Two male and two female monkeys each were assigned to 5 groups according to the design outlined in Table 2. Control animals were dosed with mannitol (particle size <210 μm) dry powder, Groups 2-4 were dosed with unmodified crystalline L-DOPA (median diameter of the particle size distribution (D50) about 50 μm), and Group 5 was dosed with particle size sifted crystalline L-DOPA such that the particle size range was 20-40 μm. Blood samples (1.6 mL per time point with sodium metabisulfite stabilizer) were collected from fasted animals pre-dose, 3, 7, 15, 30, 45, 60, 90, 120, 180, 240 and 360 minutes after dosing in all groups. Plasma was isolated from whole blood and samples were frozen prior to analysis. PK non-compartmental analysis was performed on an individual animal basis using Phoenix WinNonlin (v6.3).

Study design is summarized in Table 2.

TABLE 2
Study Design (Study 2037-003)

| | | Number of | Target | |
|---|---|---|---|---|
| | | animals | Total | |
| | | (male/ | Dose | |
| Group | Test Article | female) | (mg) | Dose regimen |
| 1 | Placebo control | 2/2 | 10 | 10 mg dose to 1 |
| | (mannitol) | | | naris |
| 2 | L-DOPA | 2/2 | 10 | 10 mg dose to 1 |
| | (crystalline, D₅₀ = | | | naris |
| | 50 μm) | | | |
| 3 | L-DOPA | 2/2 | 20 | 10 mg dose to each |
| | (crystalline, D₅₀ = | | | naris |
| | 50 μm) | | | |
| 4 | L-DOPA | 2/2 | 40 | 20 mg to each naris |
| | (crystalline, D₅₀ = | | | |
| | 50 μm) | | | |
| 5 | L-DOPA | 2/2 | 10 | 10 mg dose to 1 |
| | (crystalline, | | | naris |
| | range 20-40 μm) | | | |

The total doses achieved as well as the dose per cm² of calculated nasal surface area in each group are displayed in Table 3.

TABLE 3
Achieved L-DOPA doses in the monkey (Study 2037-003)

| | | Average | Estimated | | |
|---|---|---|---|---|---|
| | Target | body | Average | Nasal | Average |
| | dose | weight | dose | Surface Area | Dose |
| Group | (mg) | (kg) | (mg/kg) | (cm²)^{a} | (mg/cm²) |
| 2 | 10 | 3.4 | 3.0 | 16.1 | 0.62 |
| | | | | (one nostril) | |
| 3^{b} | 20 | 4.1 | 4.8 | 36.2 | 0.55 |
| | | | | (both nostrils) | |
| 4^{c} | 40 | 3.4 | 11.8 | 32.4 | 1.2 |
| | | | | (both nostrils) | |
| 5 | 10 | 3.4 | 3.0 | 16.2 | 0.62 |
| | | | | (one nostril) | |

notes:
^{a}nasal surface area (NSA) was calculated using the equation, NSA= 15.1 + 5.1 (Body Weight_{kg}) (Harris, J Aerosol Med. 2003 Summer; 16(2): 99-105) (“Harris 2003”), and the group average body weight
^{b}n = 5; male from Group 4 added to Group 3 for dose and PK analysis, as it only received one dose per nostril due to a visible nose bleed after the second spray.
^{c}n = 3, one male was removed from Group 4 and added to Group 3 for dose and PK analysis, as it only received half the intended dose.

In a few animals, struggling during dose administration led to partial delivery of the intended dose. These animals included one female in Group 2, and one male and one female in Group 3. One male in Group 4 was not administered the 2′ dose (sprays 3 and 4) in either nostril due to red discharge from the nose/muzzle. As this animal only received 1 dose to both nostrils, he was subsequently allocated to Group 3 for dose and PK analysis.

The calculated mean PK parameters are tabulated in Table 4, and the average plasma concentration-time curves are shown in FIG. 1 Following administration by the nhpPOD Device, unmodified L-DOPA delivered intranasally has dose-dependent pharmacokinetics. Further, it was observed that the small particle size may have a positive impact on the rate and extent of nasal uptake, as shown in the slight increase in AUC and Cₘₐₓ (Table 4 and FIG. 1) for Group 5 (10 mg, 20-40 μm sifted) versus Group 2 (10 mg, D₅₀ 50 μm).

TABLE 4
Mean (±SD) PK Data Following Intranasal Administration of L-
DOPA to the Monkey (n = 3-5/group) (Study 2037-003)

| Group/ | | | | Median | |
|---|---|---|---|---|---|
| No. of | | AUCₗₐₛₜ | Cₘₐₓ | Tₘₐₓ | t_{1/2} |
| animals | Dose/Formulation | (ng*min/mL) | (ng/mL) | (minute) | (minute) |
| 2, n = 4 | 10 mg, Crystalline test | 12943 ± 2707 | 51 ± 5 | 90 | 611 ± 74 |
| | article, D₅₀ 50 μm | | | | |
| 3, n = 5 | 20 mg, Crystalline test | 21820 ± 6716 | 95 ± 17 | 90 | 350 ± 170 |
| | article, D₅₀ 50 μm | | | | |
| 4, n = 3 | 40 mg, Crystalline test | 34185 ± 3441 | 150 ± 18 | 60 | 367 ± 102 |
| | article, D₅₀ 50 μm | | | | |
| 5, n = 4 | 10 mg, Crystalline | 14523 ± 3733 | 56 ± 12 | 90 | 710 ± 745 |
| | test article, particle size | | | | |
| | sifted 20-40 μm | | | | |

notes:
All control samples and pre-dose samples were below the level of quantitation (“LOQ”) of 10 ng/mL.

Following intranasal administration of unmodified crystalline L-DOPA, dose-dependent PK was observed. The earliest time point drug was measured was 3 minutes, and the median Tₘₐₓ was delayed at approximately 60-90 minutes or greater. The results shown for Group 5, where a smaller particle size L-DOPA was administered (20-40 μm), suggests that a smaller particle size may increase the rate and extent of nasal uptake and subsequent systemic exposure, as a slightly higher AUC and Cₘₐₓ was demonstrated compared to the unmodified bulk crystalline levodopa (D₅₀=50 μm) 10 mg group.

The maximum Cₘₐₓ achieved following the 40 mg dosing was 150 ng/mL. Multiple factors may contribute to this lower than expected Cₘₐₓ and longer than expected Tmax, including, e.g., chemical and physical properties of the levodopa powder, such as crystalline polymorphic state and particle size, as well as the lack of a DOPA decarboxylase inhibitor (DDC inhibitor; DDI) pre-treatment. Lastly, some monkeys in this study may have swallowed part of the dose delivered to the nasal cavity, as suggested by the second peak in the plasma concentration-time curves, which may partially be a consequence of the impact force of the propellant used in the nhpPOD Device.

5.7.1.2. Single Dose Intranasal Pharmacokinetic Study in the Cynomolgus Monkey (Non-GLP, Study 2037-004)

A single dose PK study was performed in the cynomolgus monkey, where L-DOPA dry powder (sifted or spray dried formulation) was administered intranasally using an optimized nhpPOD Device to reduce the impact of the propellant compared with the drug delivery device used in Study 2037-003.

Two male and two female monkeys each were assigned to four L-DOPA-dosed groups and one male and female were assigned to the control group according to the design outlined in Table 5. Each animal was pretreated with the DOPA decarboxylase inhibitor, benserazide, orally (size 3 capsule), receiving a 5 mg oral dose at 24, 16, 8 and 0.75 hours prior to being dosed intranasally with control material or L-DOPA. Control animals were dosed with MCC powder, Group 2 was dosed with particle size sifted crystalline L-DOPA (particle size range 20-40 μm), and Groups 3 to 5 were dosed with various excipient/spray dried formulations of L-DOPA. Blood samples (1.6 mL with sodium metabisulfite stabilizer) were collected from fasted animals pre-dose, 3, 7, 15, 30, 45, 60, 90, 120, 240, 360 and 600 minutes after dosing. Plasma was harvested from whole blood and samples were frozen prior to analysis by AIT Bioscience, Indiana, USA. Non-compartmental PK analysis was performed on an individual animal basis using Phoenix WinNonlin (v6.3).

TABLE 5
Study Design (study 2037-004)

| | | Number of | Target | |
|---|---|---|---|---|
| | | animals | Total | |
| | | (male/ | Dose | |
| Group | Test Article | female) | (mg) | Dose Regimen |
| 1 | Control | 1/1 | 20 | 10 mg once to both |
| | (microcrystalline | | | nostrils |
| | cellulose) | | | |
| 2 | L-DOPA | 2/2 | 20 | 10 mg once to both |
| | (sifted, 20-40 μm)^{a} | | | nostrils |
| 3 | L-DOPA | 2/2 | 20 | 10 mg once to both |
| | (Spray dried 1)^{b} | | | nostrils |
| 4 | L-DOPA | 2/2 | 20 | 10 mg once to both |
| | (Spray dried 2)^{c} | | | nostrils |
| 5 | L-DOPA | 2/2 | 20 | 10 mg once to both |
| | (Spray dried 3)^{d} | | | nostrils |

notes:
^{a}particle size sifted, 20-40 μm, manufactured by Teva
^{b}L-DOPA:NaCl, ratio of 98:2, manufactured by Bend Research, Oregon, USA
^{c}L-DOPA:HPMC:NaCl, ratio of 70:28:2, manufactured by Bend Research, Oregon, USA
^{d}spray dried L-DOPA manufactured by Bend Research, Oregon, USA
abbreviations:
HPMC, hydroxypropylmethyl cellulose
NaCl, sodium chloride

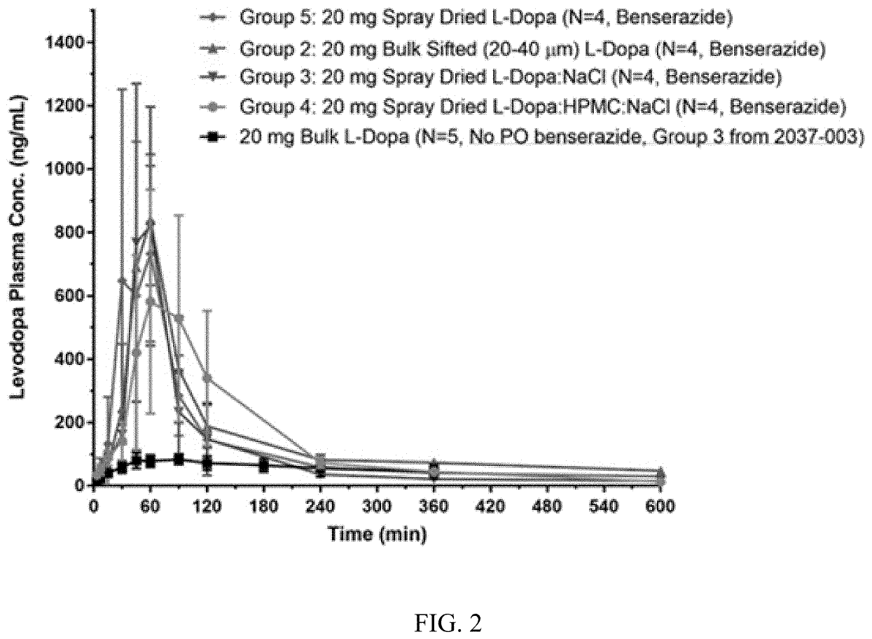

The achieved total doses and dose per cm² of calculated nasal surface area are detailed in Table 6 and the average plasma concentration-time curves are shown in FIG. 2.

TABLE 6
Achieved L-DOPA Doses in the Monkey
(n = 4/group) (study 2037-004)

| | | Average | Estimated | | |
|---|---|---|---|---|---|
| | Target | body | Average | Nasal | Average |
| | dose | weight | dose | Surface Area | Dose |
| Group | (mg) | (kg) | (mg/kg) | (cm²)^{a} | (mg/cm²) |
| 2 | 20 | 4.4 | 4.5 | 37.7 | 0.53 |
| | | | | (both nostrils) | |
| 3 | 20 | 4.0 | 5.0 | 35.4 | 0.57 |
| | | | | (both nostrils) | |
| 4 | 20 | 4.6 | 4.4 | 38.5 | 0.52 |
| | | | | (both nostrils) | |
| 5 | 20 | 4.2 | 4.7 | 36.7 | 0.54 |
| | | | | (both nostrils) | |

notes:
^{a}Nasal surface area (NSA) was calculated using the equation, NSA = 15.1 + 5.1(BW_{kg}) (Harris 2003), and the group average body weight.

Animals tolerated dosing intranasally with placebo and L-DOPA. Two L-DOPA males jerked their heads after actuation of the intranasal dose, but a complete dose was delivered. A puff of powder left the nostril of one male in Group 3 directly after administration.

The calculated mean PK parameters for all animals are shown in Table 7, and the mean plasma concentration-time curves are shown in FIG. 2. Similar pharmacokinetics were observed across the formulations containing crystalline particle size sifted L-DOPA (20-40 μm) (Group 2), spray dried L-DOPA:NaCl (Group 3), and spray dried L-DOPA (Group 5), which showed Cₘₐₓ concentrations of >900 ng/mL, well above the threshold necessary for effective treatment of ‘off’ episodes.

These Cₘₐₓ levels were significantly higher, approximately 10-fold, compared to Cₘₐₓ levels measured in the absence of the orally administered DOPA decarboxylase inhibitor, benserazide (compare Table 4). The median Tₘₐₓ observed with these formulations was 45-60 minutes, an improvement over the Tₘₐₓ observed in the absence of orally administered DOPA decarboxylase inhibitor. The spray dried L-DOPA:HPMC:NaCl formulation resulted in a slightly lower Cₘₐₓ (785 ng/mL) and longer Tₘₐₓ than the other formulations. HPMC is a commonly used excipient that increases residence time on the nasal epithelium, although these results suggest that HPMC may slow the rate of uptake of L-DOPA across the epithelium.

TABLE 7
Mean (±SD) PK Parameters for L-DOPA Following Intranasal Administration in the
Monkey (n = 4/group) with Oral Benserazide pretreatment (4 × 5 mg over 24 hours)

| | | | | Median | |
|---|---|---|---|---|---|
| | | AUCₗₐₛₜ | Cₘₐₓ | Tₘₐₓ | t_{1/2} |
| Group | Dose/Formulation | (ng*min/mL) | (ng/mL) | (minute) | (minute) |
| 2 | 20 mg, 20-40 μm bulk | 87813 ± 26577 | 1030 ± 297 | 53 | 344 ± 85 |
| | sifted | | | | |
| 3 | 20 mg, spray dried L- | 61760 ± 14987 | 962 ± 460 | 53 | 272 ± 132 |
| | DOPA:NaCl (98:2) | | | | |
| 4 | 20 mg, spray dried L- | 81446 ± 31220 | 785 ± 234 | 60 | 153 ± 47 |
| | DOPA:HPMC:NaCl | | | | |
| | (70:28:2) | | | | |
| 5 | 20 mg, spray dried L- | 76171 ± 21566 | 917 ± 358 | 45 | 230 ± 68 |
| | DOPA | | | | |

Abbreviations:
HPMC, hydroxypropylmethyl cellulose;
NaCl, sodium chloride

In summary, the maximum mean plasma level achieved was 1,030 ng/mL following delivery of 20 mg crystalline particle size sifted L-DOPA (Teva), although two of the spray dried formulations, L-DOPA:NaCl and L-DOPA (Bend) achieved similar Cₘₐₓ levels (>900 ng/mL). Improved (faster) Tₘₐₓ values (45-60 min) were observed in this study for all L-DOPA formulations tested compared to L-DOPA administered in the absence of the oral DOPA decarboxylase inhibitor, benserazide (>90 min; study 2037-003).

Exposure levels (AUC) increased 3-to 4-fold when L-DOPA was administered by an optimized nhpPOD Device with oral benserazide pretreatments (5 mg×4 doses over 24 hours), and overall the large AUC and long half-life for all groups suggest reasonable absorption of L-DOPA across the nasal epithelium regardless of formulation tested in this study.

The control group male had no measurable L-DOPA LOQ of 10 ng/mL) in plasma samples collected at any time point. The control group female, however, did have low levels of L-DOPA in plasma samples collected from 3 to 120 minutes (12.7-20.3 ng/mL). This was considered likely to be due to low endogenous levels of L-DOPA.

5.7.1.3. Single Dose Intranasal Pharmacokinetic Study in the Cynomolgus Monkey (non-GLP, study 2037-006)

A third single dose PK study was performed in the cynomolgus monkey, where L-DOPA dry powder (spray dried formulations) were administered intranasally using an nhpPOD Device. Two male and two female monkeys each were assigned to five groups, of which only four are described here. Each group was administered a different spray dried formulation of L-DOPA, according to the design outlined in Table 8. Each animal was pretreated with oral benserazide (size 3 capsule) such that each animal in Groups 1-4 received a 5 mg dose at 24, 16, 8 and 0.75 hours prior to being dosed intranasally with L-DOPA. The Groups 2 and 3 test product had a slight difference in the manufacturing process (different L-DOPA starting material particle size), but otherwise the formulations tested were the same.

Blood samples (1.6 mL stabilized with sodium metabisulfite) were collected from fasted animals pre-dose, 3, 7, 15, 30, 45, 60, 90, 120, 240, 360 and 600 minutes after dosing from animals in all groups. Plasma was isolated from whole blood and samples were frozen prior to analysis by AIT Bioscience, Indiana, USA. Non-compartmental PK analysis was performed on an individual animal basis using Phoenix WinNonlin (v6.3).

TABLE 8
Study Design (study 2037-006)

| | | | Target | |
|---|---|---|---|---|
| | | Number of | Total | |
| | | animals | Dose | |
| Group | Test Article | (M/F) | (mg) | Dose Regimen |
| 1 | L-DOPA:NaCl:HPMC:DSPC | 2/2 | 20 | 10 mg once to both |
| | (68:2:16:14) | | | nostrils |
| 2 | L-DOPA:NaCl:HPMC:DSPC | 2/2 | 20 | 10 mg once to both |
| | (68:2:23:7) | | | nostrils |
| 3 | L-DOPA:NaCl:HPMC:DSPC | 2/2 | 20 | 10 mg once to both |
| | (68:2:23:7) | | | nostrils |
| 4 | L-DOPA:NaCl:HPMC:Maltoside | 2/2 | 20 | 10 mg once to both |
| | (68:2:23:7) | | | nostrils |

Abbreviations:
DSPC, 1,2-distearoyl-sn-glycero-3-phosphocholine;
F, female;
HPMC, hydroxypropyl methyl cellulose;
M, male;
NaCl, sodium chloride;
Maltoside, n-dodecyl-β-D-maltopyranoside

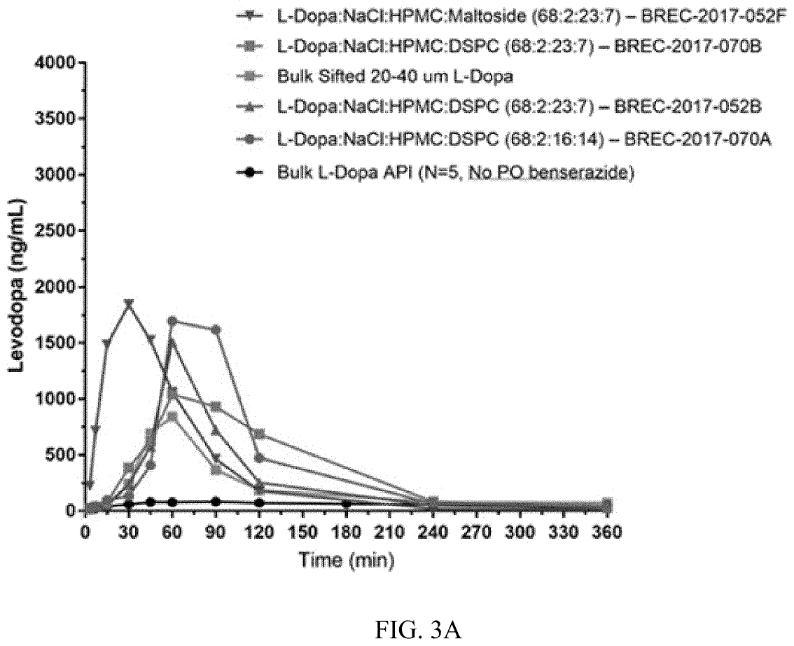

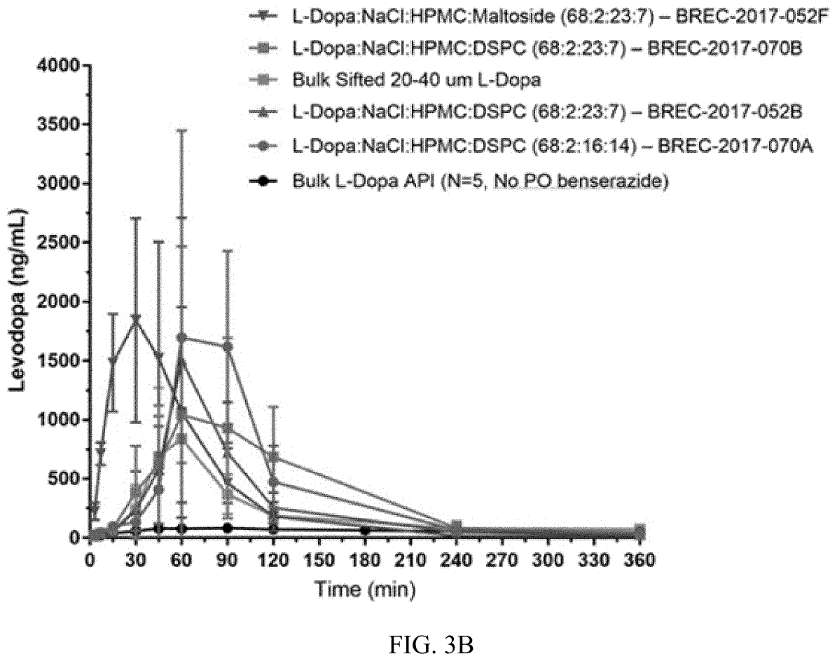

Results are displayed in Table 9 and FIGS. 3A-3B. All formulations tested achieved similar or up to 1.7-fold greater total exposure (AUC) and increased Cmax, up to 2.3-fold, compared to the spray dried formulations tested in the second PK study (study 2037-004, described above). The measured Tₘₐₓ values for the groups containing L-DOPA and HPMC/DSPC all had similar or greater values compared to the formulations tested in the previous study. Surprisingly, however, the Tₘₐₓ for the maltoside formulation was significantly shorter, with the median Tₘₐₓ observed at 30 min, and all 4 monkeys in this group achieved plasma L-DOPA concentrations >400 ng/mL within 7 minutes following L-DOPA administration by the nhpPOD Device. As the goal of this drug device combination product is to achieve plasma concentrations of L-DOPA that are effective to very quickly to switch a patient from ‘off’ to ‘on’, the formulation containing maltoside was selected for testing in the human clinical trial described in Example 2 below.

TABLE 9
Mean (±SD) PK Parameters for L-DOPA Following Intranasal Administration
in the Monkey (n = 4/group) with Pre-treatment with Oral
Benserazide (4 × 5 mg capsule over 24 hours for Groups 1-4)

| | | | | Median | |
|---|---|---|---|---|---|
| | | AUCₗₐₛₜ | Cₘₐₓ | Tₘₐₓ | t_{1/2} |
| Group | Dose/Formulation | (ng · min/mL) | (ng/mL) | (minute) | (minute) |
| 1 | L-DOPA:NaCl:HPMC:DSPC | 150440 ± 80177 | 2395 ± 1129 | 75 | 231 ± 48 |
| | (68:2:16:14) | | | | |
| 2 | L-DOPA:NaCl:HPMC:DSPC | 136449 ± 11181 | 1810 ± 1031 | 105 | 139 ± 39 |
| | (68:2:23:7) | | | | |
| 3 | L-DOPA:NaCl:HPMC:DSPC | 98999 ± 35043 | 1817 ± 863 | 60 | 165 ± 51 |
| | (68:2:23:7) | | | | |
| 4 | L-DOPA:NaCl:HPMC:Maltoside | 127059 ± 67663 | 1880 ± 844 | 30 | 49 ± 23 |
| | (68:2:23:7) | | | | |

Abbreviations:
DSPC, 1,2-distearoyl-sn-glycero-3-phosphocholine;
HPMC, hydroxypropyl methyl cellulose;
NaCl, sodium chloride;
Maltoside, n-dodecyl-β-D-maltopyranoside

5.7.1.4. Single Dose Intranasal Pharmacokinetic Study in the Cynomolgus Monkey (Non-GLP, Research Study Number 2037-007)

A fourth single dose PK study was performed in the cynomolgus monkey. L-DOPA dry powder (spray dried) formulations were administered intranasally using an nhpPOD Device. Ten male and ten female monkeys were assigned to five groups. Each group was administered a different spray dried formulation of L-DOPA, according to the design outlined in Table 10. Each animal was pretreated with oral benserazide (size 3 capsule) such that each animal in Groups 1-5 received a 5 mg oral dose at 24, 16, 8 and 0.75 hr prior to being dosed intranasally with L-DOPA.

Blood samples (1.6 mL stabilized with sodium metabisulfite) were collected from fasted animals pre-dose, 3, 7, 15, 30, 45, 60, 90, 120, 240, 360 and 600 minutes after dosing from animals in all groups. Plasma was isolated from whole blood and samples were frozen prior to analysis by AIT Bioscience, Indiana, USA. Non-compartmental PK analysis was performed on an individual animal basis.

TABLE 10
Study Design (study 2037-007)

| | | | Target | |
|---|---|---|---|---|
| | | Number of | Total | |
| | | animals | Dose | |
| Group | Test Article | (M/F) | (mg) | Dose Regimen |
| 1 | L-DOPA:NaCl:HPMC:Maltoside | 2/2 | 20 | 10 mg once to both |
| (FS-A-HQ00001) | (68:2:29:1) | | | nostrils |
| 2 | L-DOPA:NaCl:HPMC:Maltoside | 2/2 | 20 | 10 mg once to both |
| (FS-B-HQ00002) | (68:2:29:1) | | | nostrils |
| 3 | L-DOPA:NaCl:HPMC:Maltoside | 2/2 | 20 | 10 mg once to both |
| (FS-C-HQ00003) | (68:2:29:1) | | | nostrils |
| 4 | L-DOPA:NaCl:HPMC:Maltoside | 2/2 | 20 | 10 mg once to both |
| (FS-D-HQ00004) | (68:2:29.9:0.1) | | | nostrils |
| 5 | L-DOPA:NaC:HPMC:Maltoside | 2/2 | 20 | 10 mg once to both |
| (FS-E-HQ00005) | (68:2:29.5:0.5) | | | nostrils |

Abbreviations:
F, female;
HPMC, hydroxypropyl methyl cellulose;
M, male;
NaCl, sodium chloride;
Maltoside, n-dodecyl-β-D-maltopyranoside (DDM)

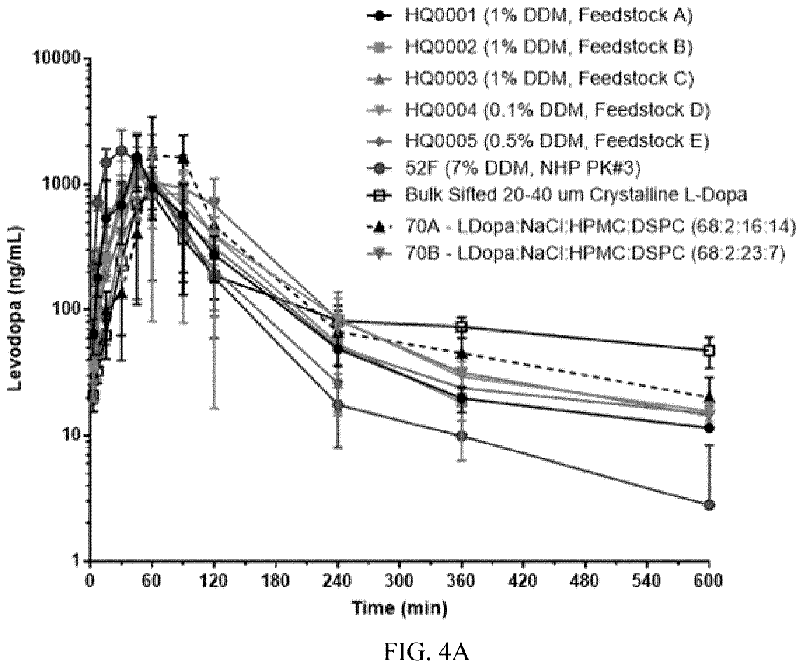

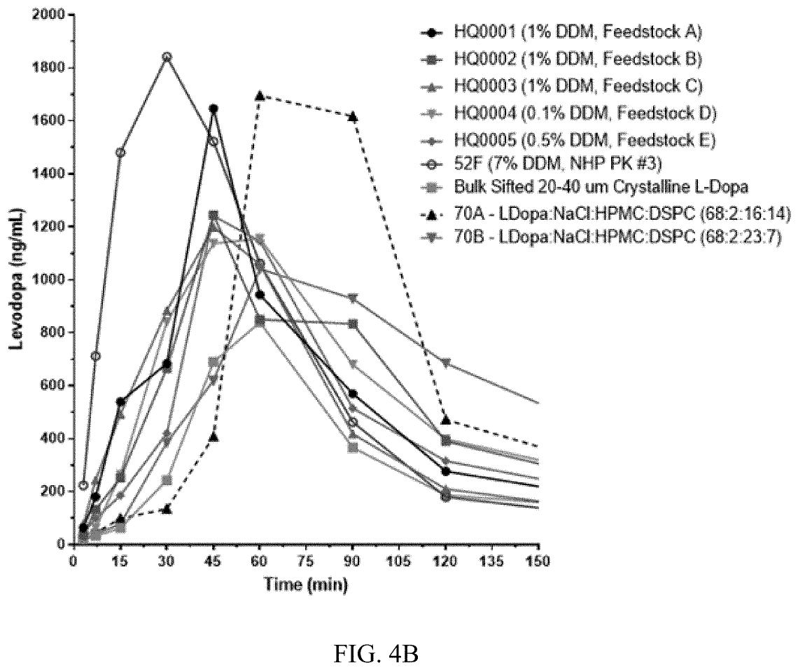

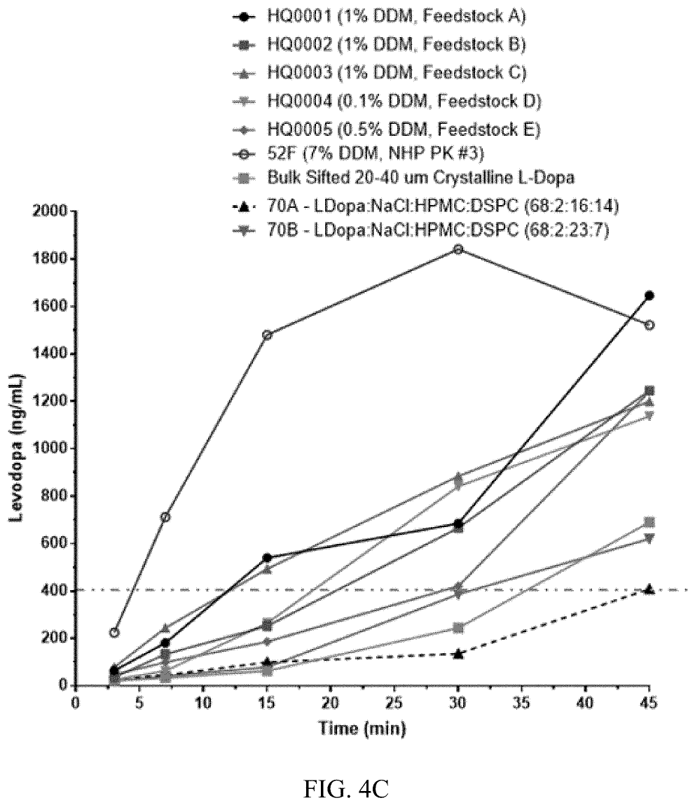

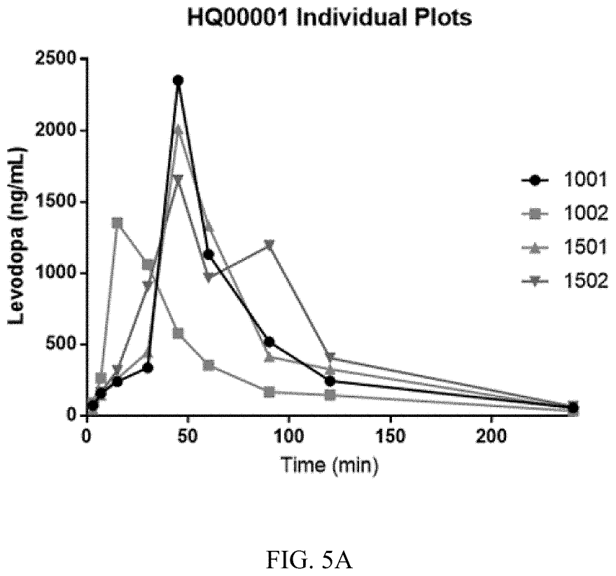

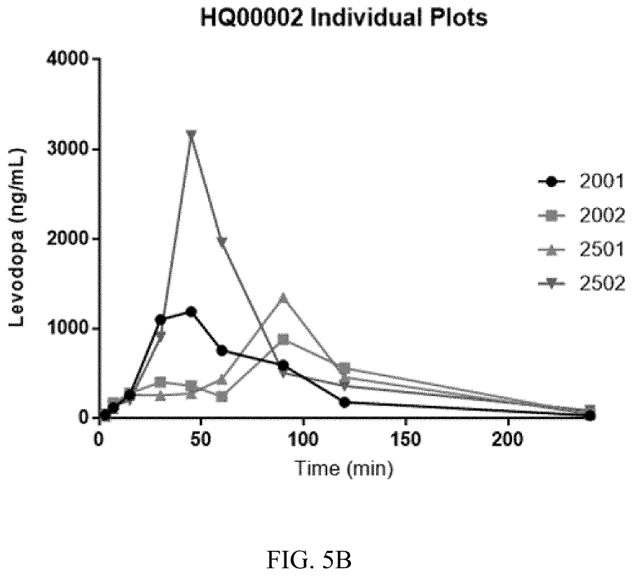

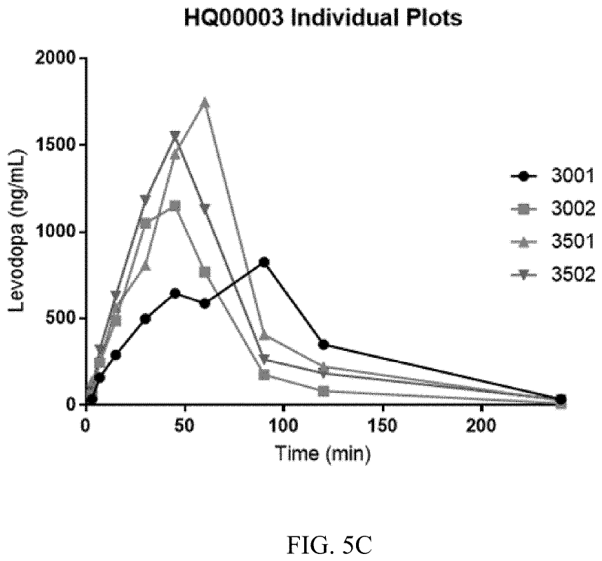

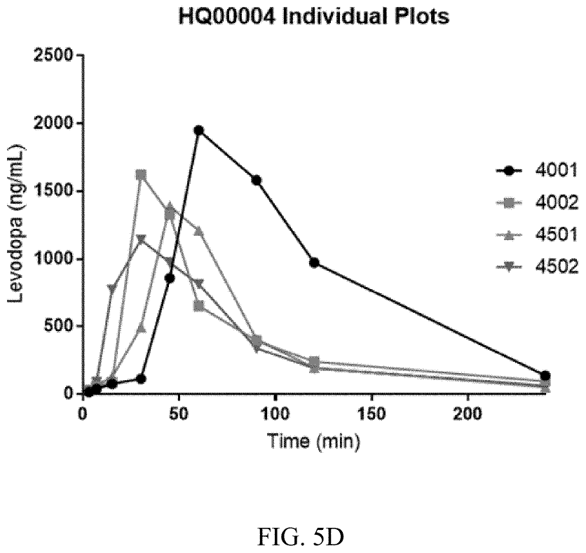

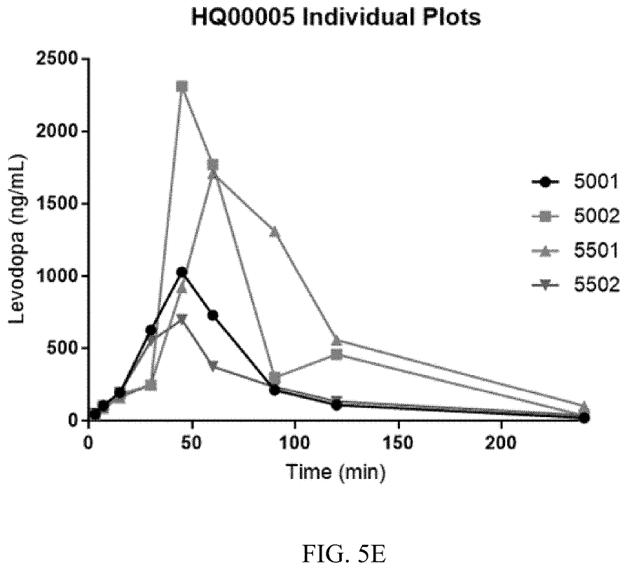

Results are displayed in Table 11 and FIGS. 4A-C and FIGS. 5A-E. All the tested formulations achieved total exposure (AUC), Cₘₐₓ and Tₘₐₓ values similar to the spray dried formulations tested in the third PK study (study 2037-006, described above) as provided in FIGS. 4A-C. These formulations have similar or up to 1.7-fold greater total exposure (AUC) and increased Cₘₐₓ, up to 2.3-fold, compared to the spray dried formulations tested in the second PK study (study 2037-004, described above). Although the third PK study demonstrated that the Tₘₐₓ for the formulation including 7% maltoside is significantly shorter than the Tₘₐₓ for the formulation including DSPC instead of maltoside, Tₘₐₓ values for the formulations including different concentrations (0.1, 0.5, 1%) of maltoside were not significantly different from each other.

TABLE 11
Mean (±SD) PK Parameters for L-DOPA Following Intranasal Administration in the Monkey (n =
4/group) and Pre-treatment with Oral Benserazide (4 × 5 mg capsule over 24 hours for Groups 1-5)

| | | | | Median | |
|---|---|---|---|---|---|
| Group | | | | Tₘₐₓ | |
| (formulation | | AUCₗₐₛₜ | Cₘₐₓ | (minute) | t_{1/2} |
| batch) | Dose/Formulation | (ng · min/mL) | (ng/mL) | [min, max] | (minute) |
| 1 | L-DOPA:NaCl:HPMC:Maltoside | 110,786 ± 30,681 | 1840 ± 434 | 45 | 98 ± 42 |
| (FS-A- | (68:2:29:1) | | | [15, 45] | |
| HQ00001) | | | | | |
| 2 | L-DOPA:NaCl:HPMC:Maltoside | 113,551 ± 33,367 | 1643 ± 1024 | 68 | 55 ± 12 |
| (FS-B- | (68:2:29:1) | | | [45, 90] | |
| HQ00002) | | | | | |
| 3 | L-DOPA:NaCl:HPMC:Maltoside | 92,404 ± 18,094 | 1310 ± 413 | 53 | 42 ± 6 |
| (FS-C- | (68:2:29:1) | | | [45, 90] | |
| HQ00003) | | | | | |
| 4 | L-DOPA:NaCl:HPMC:Maltoside | 125,947 ± 53,361 | 1525 ± 345 | 37.5 | 106 ± 50 |
| (FS-D- | (68:2:29.9:0.1) | | | [30, 60] | |
| HQ00004) | | | | | |
| 5 | L-DOPA:NaCl:HPMC:Maltoside | 101,243 ± 52,699 | 1438 ± 717 | 45 | 80 ± 39 |
| (FS-E- | (68:2:29.5:0.5) | | | [45, 60] | |
| HQ00005) | | | | | |

Abbreviations:
F, female;
HPMC, hydroxypropyl methyl cellulose;
M, male;
NaCl, sodium chloride;
Maltoside, n-dodecyl-β-D-maltopyranoside

5.7.1.5. Materials and Methods

Materials and methods for the studies described above are described here.

5.7.1.5.1. Summary

TABLE 12

| | |
|---|---|
| Nasal Administration Device: | Part Name: nhpPOD Device |
| | (POD Device, NHP, Powder, study 2037-003, 2037-004, 2037- |
| | 006, 2037-007) |
| | Study #2037-003: Part Number: 00308-01 |
| | Description: powder delivery using the nhpPOD with reducer |
| | and extension tube |
| | Study #2037-004: Part Number: 00308-02 |
| | Description: powder delivery using the nhpPOD with |
| | optimized reducer and extension tube |
| | Study #2037-006: Part Number: 00308-02 |
| | Description: powder delivery using the nhpPOD with |
| | optimized reducer and extension tube |
| | Study #2037-007: Part Number: 00308-02 |
| | Description: powder delivery using the nhpPOD with |
| | optimized reducer and extension tube |
| Preparation Details: | The control and test articles were received from the Sponsor, and |
| | loaded into the powder nhpPOD (powder non-human primate |
| | Precision Olfactory Delivery) Device tip on the day of dosing. |
| | The nhpPOD Device tip was tapped on the powder, Levodopa |
| | formulation test article or control, to load 10 mg of powder into |
| | each device tip and excess powder was wiped from the tip using a |
| | Kimwipe. |
| | Standard laboratory procedures were used and no problems were |
| | encountered. |
| Dose Administration Details: | Animals were dosed while being held in the prone position with |
| | the head in a neutral position and sight line parallel to the ground |
| | (horizontal plane). |

5.7.1.5.2. nhpPOD devices

The nhpPOD device described in section 5.3.4.4 and FIGS. 9A-E was used to conduct the studies in Table 12 above.

5.7.1.5.3. Methods

Bioanalysis of NHP Plasma Samples for Levodopa

A non-GLP bioanalytical method was developed for analysis of levodopa in NHP plasma at AIT Bioscience (Indianapolis, Ind., USA). This method was based on a validated method for the quantitation of levodopa in rat plasma, previously developed and validated at AIT Bioscience for Impel.

Preparation of Plasma Samples for Analysis of Levodopa

Sodium metabisulfite (4% by volume of a 100 mg/mL solution in sterile water) was added as stabilizer (e.g. 10.4 μL of the 100 mg/mL sodium metabisulfite solution was added to 250 μL of blood) within a few minutes after each blood collection followed by thorough, gentle mixing by inversion prior to being placed on wet ice. The tubes were kept protected from light (i.e. in a closed cooler and/or covered with aluminum foil) and generally centrifuged within 15 minutes of collection. Samples were centrifuged under refrigeration (set to +4° C. and 1500 g RCF) for targeted 10 minutes. Plasma was recovered, transferred using a micropipette into separate tubes and placed on dry ice, pending storage in a freezer set to maintain −70° C. until shipment.

Preparation of Calibration Standards and Quality Control Samples

Stock solutions of levodopa were prepared to 2.00 mg/mL in 0.1N perchloric acid and stored in amber glass at 2-8° C.

K₂EDTA fortified NHP plasma was prepared by mixing 100 mg/mL aqueous sodium metabisulfite with NHP plasma in a 4:96 ratio.

Calibration Standard (CS) spiking solutions (100,000 ng/mL to 200 ng/mL) were prepared by dilution of a stock solution with 100 mg/mL sodium metabisulfite solution. CS were then prepared by diluting these spiking solutions with K2EDTA fortified NHP plasma in a 5:95 ratio to achieve nominal concentrations of 5,000 to 10.0 ng/mL, in 8 levels.

QC spiking solutions were similarly prepared by dilution of a separate stock solution with 100 mg/mL sodium metabisulfite solution. QC were then prepared by diluting these spiking solutions with K2EDTA fortified NHP plasma in a 5:95 ratio to achieve nominal concentrations of 3,750, 300, 30, and 10.0 ng/mL.

CS and QC pools were prepared and sub-divided into single-use aliquots stored in polypropylene vials at −80° C. Aliquots of the CS and QC pools were thawed for one-time use on wet ice.

A sample volume of 50.0 μL was aliquoted into a 1.2 mL 96-well plate and mixed with 25.0 μL internal standard solution (2000 ng/mL L-DOPA-2,5,6-D3 in 2N perchloric acid). Then, 125 μL of water was added to each well. The plates were covered and the mixtures were vigorously shaken, vortexed to mix, and centrifuged. Using a Tomtec Quadra96 liquid handler, a 100 μL aliquot of the supernatant was transferred to a clean 96-well plate for LC-MS/MS injection.

Samples were analyzed on a Waters Acquity liquid chromatograph interfaced with a Thermo Scientific TSQ Vantage triple quadrupole mass spectrometer with ESI ionization. Each extracted sample was injected (10.0 μL) onto an Acquity HSS C18 column (2.1×50.0 mm; 1.8 μm) equilibrated at 30° C. Mobile Phase A was 100-0.1 water-formic acid. Mobile Phase B was 100-0.1 acetonitrile-formic acid.

The LC gradient is tabulated in Table 13 below.

TABLE 13

| Time | Flow Rate | Mobile Phase A | Mobile Phase B |
|---|---|---|---|
| (min) | (mL/min) | (%) | (%) |
| 0.00 | 0.500 | 100.0 | 0.0 |
| 1.00 | 0.500 | 95.0 | 5.0 |
| 1.70 | 0.500 | 88.0 | 12.0 |
| 2.00 | 0.500 | 88.0 | 12.0 |
| 2.25 | 0.500 | 30.0 | 70.0 |
| 3.25 | 0.500 | 30.0 | 70.0 |
| 3.50 | 0.500 | 100.0 | 0.0 |
| 6.00 | 0.500 | 100.0 | 0.0 |

The retention time, mass transition and precursor charge state for each compound are as follows:

TABLE 14

| | Expected | | Product | |
|---|---|---|---|---|
| | Retention | Precursor | Observed | Charge State |
| | Time | Mass/Charge | Mass/Charge | of Precursor |
| Compound | (min) | (m/z) | (m/z) | Ion |
| Levodopa | 0.68 | 198.127 | 152.071 | +1 |
| Levodopa-2,5,6- | 0.68 | 201.141 | 154.096 | +1 |
| D3 | | | | |

Peak area ratios from the calibration standard responses were regressed using a (1/concentration²) linear fit for levodopa. The regression model was chosen based upon the behavior of the analyte across the concentration range used during method development.

Pharmacokinetic Parameter Calculations and Data Analysis

Plasma concentration-time data for levodopa was used to determine pharmacokinetic (PK) parameters. Non-compartmental analysis (NCA) was performed on the individual subject plasma concentration data using the software Phoenix WinNonlin (v6.3).

The following pharmacokinetic parameters were determined: Cₘₐₓ, Tₘₐₓ, Tₗₐₛₜ, AUCₗₐₛₜ, and t_{1/2} where possible. Various additional pharmacokinetic parameters were automatically generated by Phoenix WinNonlin software but were not presented in this report. The following configuration was used for the analysis:

Model type selection (Plasma 200-202) was based on the biological matrix (plasma) and the dose type was based on the route of administration (extravascular). Observed parameters were used for the analysis. The acceptance criteria for Kₑₗ determination were regression of at least three time points in the apparent terminal elimination phase, excluding Cₘₐₓ, otherwise t_{1/2} was not determined or reported. Nominal blood sampling times and nominal dose levels were used. Concentrations reported as below the lower limit of quantification were treated as zero (0).

5.7.2. Example 2: Phase IIa, Randomized, Double Blind, Placebo Controlled, Single Ascending Dose, Safety and Pharmacokinetic/Pharmacodynamic Study of INP103 (POD L-DOPA) Administered in the Presence of Benserazide to Levodopa Responsive Parkinson's Disease Patients

5.7.2.1. Study Design

A powder formulation of L-DOPA (levodopa) was tested in a randomized, double-blind, placebo controlled, single ascending dose study to demonstrate safety, tolerability and PK/pharmacodynamics of L-DOPA delivered by the 1231 Precision Olfactory Delivery (“POD®”) device to human subjects. The 1231 POD device is a handheld, manually actuated, propellant-driven, metered-dose administration device intended to deliver a powder drug formulation to the nasal cavity.

Intranasal administration was performed with single ascending doses of one (35 mg), two (70 mg) or four (140 mg) administrations (puffs) of L-DOPA into the naris. L-DOPA was administered 60 minutes after oral benserazide hydrochloride 25 mg. An inert, visually similar product without L-DOPA (microcrystalline cellulose) was administered as a placebo.

L-DOPA responsive Parkinson's disease patients were enrolled in the study. The subjects were males or females between 40 and 80 years of age, diagnosed with idiopathic Parkinson's disease, and prone to and able to recognize OFF episodes when their usual medication has worn off. For enrollment, they must have been shown to be responsive to L-DOPA medication showing more than 30% improvement in MDS-UPDRS Part III Motor Examination score.

All of the subjects received oral benserazide hydrochloride (benserazide) 25 mg on arrival at the research site (and 60±5 minutes prior to L-DOPA or placebo dosing) and the time recorded. The subjects were divided into three cohorts and each cohort was treated as follows. Cohort 1: Each subject in this cohort received one dose of 35 mg of L-DOPA or placebo delivered by one actuation of the POD device. Cohort 2: Each subject in this cohort received two 35 mg doses of L-DOPA or placebo delivered by two actuations of the POD device, for a total of 70 mg of L-DOPA or placebo. Cohort 3: Each subject in this cohort received four 35 mg doses of L-DOPA or placebo delivered by four actuations of the POD device, for a total of 140 mg of L-DOPA or placebo.

Safety and tolerability, pharmacokinetics and pharmacodynamics of intranasally delivered L-DOPA were assessed in the subjects as described below.

Safety and Tolerability Assessments: Specific assessments to evaluate treatment safety included the following: overall dyskinesia assessment, nasal inspection (as part of physical examinations), the frequency and type of AEs, concomitant medications (including any short acting anti-OFF medication, permissible only at/after 120 minutes post dosing on dosing days alongside the subject's delayed usual anti-PD morning dose), clinical laboratory testing, 12-lead ECGs and vital signs (to include supine and standing blood pressure, all other vital signs supine only). All treated subjects were observed for 240 minutes post dose and underwent follow-up evaluations (by appropriately trained/qualified staff) at Day 7.

Pharmacokinetic Assessments: PK blood samples were collected (recommended to be from an indwelling cannula positioned so that it does not interfere with arm movements) within 15 minutes prior to dosing and at 30, 60, 90 and 120 minutes after dosing (with L-DOPA).

Pharmacodynamics Assessments: Measurement of a full MDS-UPDRS score was conducted at the start of all visits. Changes from baseline in MDS-UPDRS Part III scores were estimated using a Mixed Model for Repeated Measures (MMRM) with treatment group (L-DOPA 35 mg, 70 mg, or 140 mg, or placebo), time point (15, 30, 45, 60, 90 or 120 minutes) and the interaction between treatment group and time point as fixed factors.

Dyskinesia assessment, nasal inspection, laboratory evaluations, vital signs assessments (including supine and standing blood pressure, all other vital signs supine only) and ECG parameters showed no significant difference between the subjects treated with L-DOPA and placebo. The results demonstrate that L-DOPA delivered by the POD is safe and tolerable.

L-DOPA concentrations in the PK blood samples were summarized with descriptive statistics (arithmetic and geometric mean, SD, median, minimum, and maximum) by treatment group and time point. In addition, PK parameters (AUC₀₋₂ₕ, Cₘₐₓ, Tₘₐₓ) were summarized with descriptive statistics by treatment group.

5.7.2.2. Study Formulation

The study drug was a spray-dried formulation containing L-DOPA:NaCl:HPMC: Maltoside in the weight ratios of 68:2:29:1 (INP103).

5.7.2.3. Study Results

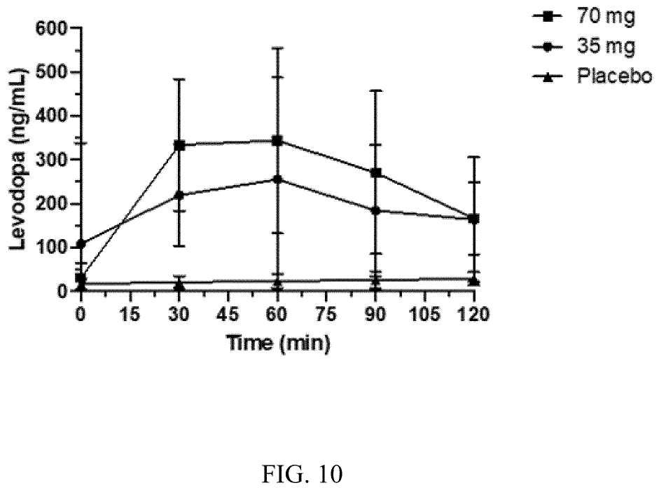

An interim analysis of data from cohorts 1 and 2, with partial data read-out, demonstrated that INP103 was well tolerated. Interim pharmacokinetic data for cohort 1 (35 mg) and cohort 2 (70 mg) are shown in FIG. 10 and show that the PK of INP103 at the 70 mg dose reached blood concentrations in the range known to be effective to treat Parkinson OFF episodes with a mean time to Cₘₐₓ (Tₘₐₓ) of 30-60 minutes.

### 6. INCORPORATION BY REFERENCE

The disclosures of each and every patent, patent application, and publication cited herein are hereby incorporated by reference in their entirety.

### 7. EQUIVALENTS

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of treating an OFF episode in a patient with Parkinson's disease (PD) or a Parkinson syndrome, the method comprising:
administering to a subject suffering from an OFF episode of either Parkinson's disease or a Parkinson syndrome an effective dose of levodopa (L-DOPA) by delivering a dry pharmaceutical composition comprising levodopa (L-DOPA) using an intranasal delivery device, thereby providing:
(a) a mean peak plasma levodopa concentration (Cₘₐₓ) of at least 200 ng/mL, with
(b) a mean time to Cₘₐₓ (Tₘₐₓ) of levodopa of less than or equal to 60 minutes, wherein the intranasal delivery device is configured to deliver the dry pharmaceutical composition.

2. The method of claim 1, wherein the mean peak plasma levodopa concentration (Cₘₐₓ) provided by the dose is at least 400 ng/mL.

3. The method of claim 1, wherein the intranasal administration of levodopa is adjunctive to oral administration of a DDI, optionally wherein the oral DDI is benserazide or carbidopa.

4. The method of claim 3, wherein the intranasal administration of levodopa is adjunctive to oral treatment with a DDI and oral treatment with levodopa, optionally wherein the oral treatment with a DDI and oral treatment with levodopa is with an oral dosage form containing a fixed dose combination of a DDI and levodopa.

5. The method of claim 1, wherein the patient has PD or a Parkinson syndrome selected from the group consisting of post-encephalitic parkinsonism, symptomatic parkinsonism following carbon monoxide intoxication, or symptomatic parkinsonism following manganese intoxication.

6. The method of claim 1, wherein the dry pharmaceutical composition is a powder, optionally wherein the median diameter of the levodopa particle size distribution (D50) in the powder is 5 μm-500 μm, 5 μm-250 μm, 5 μm-100 μm, 5 μm-75 μm, 5 μm-50 μm, 10 μm-50 μm or 20 μm-40 μm.

7. The method of claim 1, wherein the dry pharmaceutical composition comprises levodopa (i) in a crystalline form, (ii) in an amorphous form, optionally wherein the amorphous form is obtained by spray-drying, or (iii) in a partially crystalline and partially amorphous form.

8. The method of claim 1, wherein the dry pharmaceutical composition comprises no more than 80 wt % levodopa, 50-80 wt % levodopa, 50-70 wt % levodopa, or 65-70 wt % levodopa.

9. The method of claim 1, wherein the dry pharmaceutical composition further comprises a nonionic surfactant, optionally wherein the nonionic surfactant is an alkyl maltoside, optionally wherein the alkyl maltoside is n-dodecyl β-D-maltoside.

10. The method of claim 1, wherein the dry pharmaceutical composition further comprises HPMC.

11. The method of claim 1, wherein the dry pharmaceutical composition further comprises a salt of a monovalent inorganic cation, optionally wherein the salt is NaCl.

12. The method of claim 1, wherein the dry pharmaceutical composition comprises 68 wt % levodopa, 2 wt % NaCl, 29 wt % HPMC, and 1 wt % n-dodecyl β-D-maltoside, optionally wherein the composition is a spray dried composition.

13. The method of claim 1, wherein the effective dose is a dose of dry pharmaceutical composition containing levodopa in an amount effective to reverse an OFF episode within 60 minutes.

14. The method of claim 13, wherein the effective dose of levodopa is 25-150 mg, 35 -140 mg, 35 mg, 70 mg, 105 mg, or 140 mg.

15. The method of claim 1, wherein the effective dose is administered (i) as a single undivided dose or (ii) as a plurality of equally divided sub-doses.

16. The method of claim 1, wherein the intranasal delivery device is (i) a handheld, manually actuated, metered-dose intranasal administration device or (ii) a manually actuated, propellant-driven, metered-dose intranasal administration device.

17. The method of claim 16, wherein the levodopa composition is, prior to device actuation, (i) encapsulated within a capsule present in the device or (ii) stored within a dose container that is removably coupled to the device.
